(19) United States
(12) Patent Application Publication
(10) Pub. No.: US 2006/0292095 A1
(43) Pub. Date: Dec. 28, 2006

(54) Gelled oil particles comprising at least one hydrophobic sunscreen

(75) Inventors: Bruno Biatry, Vincennes (FR); Jean-Thierry Simonnet, Cachan (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/434,731

(22) Filed: May 17, 2006

(30) Foreign Application Priority Data
May 17, 2005 (FR) 05 51270

(51) Int. Cl. A61K 8/81; A61K 8/73

(52) U.S. Cl. 424/59

(57) ABSTRACT

The present invention relates to calibrated oily particles comprising at least one hydrophobic sunscreen and comprising at least one oily phase structured with at least one gelling polymer, characterized in that it has a mean size of less than or equal to 20 μm, in that the said structured oily phase has a melting point of greater than or equal to 40° C. and in that their circularity index is between 0.9 and 1.

### CROSS-REFERENCE TO RELATED APPLICATION

This non provisional application claims the benefit of French Application No. 05 51270 filed on May 17, 2005, the disclosure of which is incorporated herein by reference in its entirety.

### BACKGROUND

The present invention relates to calibrated, spherical oily particles comprising a hydrophobic sunscreen.

The present invention also relates to a cosmetic or dermatological composition comprising these particles, and also to a process for manufacturing these particles.

These particles are useful in particular as agents for increasing the sun protection factor (SPF).

It is known that light radiation with wavelengths of between 280 nm and 400 nm permits tanning of the human epidermis and that rays with wavelengths of between 280 nm and 320 nm, which are known as UV-B, cause erythema and skin burns that can harm the development of a natural tan.

It is also known that UV-A rays, with wavelengths of between 320 nm and 400 nm, which cause tanning of the skin, are liable to induce impairment therein, especially in the case of sensitive skin or of skin that is continually exposed to sunlight. UV-A rays in particular cause a loss of elasticity of the skin and the appearance of wrinkles, leading to prematurely aged skin. They promote the onset of the erythemal reaction or amplify this reaction in certain individuals and may even be the cause of phototoxic or photoallergic reactions.

Various types of sunscreen exist on the market for screening out UVA and UVB rays: pigments and chemical screening agents (or organic UV-screening agents). These sunscreens must be able to absorb or block the harmful rays of the sun while at the same time remaining harmless to the user.

The object of the development of compositions comprising a sunscreen is generally to obtain the best ratio in terms of sunscreen content/efficacy. To do this, “SPF boosters” are very regularly used, for instance waxes or fatty-phase-gelling polymers. However, these compounds increase the viscosity of the fatty phase as a whole and, as a result, that of the final emulsion. It is then impossible to obtain fluid and vaporizable suspensions. Furthermore, these technical solutions condition the emulsification at high temperature of the composition as a whole, which proves to be detrimental to the heat-sensitive active agents that may be present in the compositions for photoprotection.

The efficacy of an antisun composition for the skin is generally reflected in terms of the sun protection factor (SPF), which is defined as the ratio of the amount of energy required to induce the onset of erythema on skin protected with the agent for screening out UV radiation, to the amount of energy required to induce the onset of erythema on unprotected skin.

Particles of oily nature have been proposed as an alternative.

Thus, the use of particles with a diameter of about from 50 μm to 10 mm and comprising an oil structured with a gelling agent to formulate a composition for cleansing the skin and the hair has been proposed (see U.S. Pat. No. 6,737,394).

WO 02/092043 discloses a skincare composition comprising an aqueous phase in which is dispersed an oily phase structured with a gelling agent. The oily phase, whose viscosity does not exceed 5000 Pa·s, is dispersed therein in the form of particles ranging from 1 to 500 μm in size.

EP 0 375 520 discloses a cosmetic composition for topical application comprising particles of fatty substance, comprising an active product, and having a diameter of between 3 and 10 μm.

However, once again, the particles described above as a whole do not propose active substances of sunscreen type and/or are obtained by means of processes that do not allow the production of particles that have in one instance been calibrated in a predetermined size range.

Moreover, compositions more particularly intended for protecting the skin and/or the hair against UV radiation are described in EP 1 331 000. These compositions comprise at least one liquid fatty phase, at least one organic UV-screening agent and at least one semi-crystalline polymer that is solid at room temperature, with a melting point of less than 70° C. However, no gelled and calibrated oily particles, and no dispersion of the said gelled and calibrated oily particles, are disclosed and even less so is their value in terms of improving the SPF.

Documents US 2004/0042980 and US 2004/0247549 describe emulsions containing an oily-phase-structuring polymer, preferably of alkyl polyamide type, and also containing a sunscreen. In the same manner as for the document cited above, no mention is made of gelled and calibrated oily particles.

### SUMMARY

There is consequently a need to optimize the deposition of sunscreens on the skin and/or the hair in order to improve the sun protection factor (SPF).

There is also a need to obtain structured oily particles comprising a sunscreen, which are stable and which do not exude over time.

There is also a need to obtain oily particles comprising a sunscreen that are homogeneous in their structure and their size distribution.

There is also a need to obtain oily particles comprising a sunscreen whose integrity is maintained during application to a support.

There is also a need to obtain compositions comprising a sunscreen, which also comprise heat-sensitive active agents, these heat-sensitive active agents not needing to undergo a step of high-temperature emulsification of the composition as a whole.

Finally, there is a need to obtain compositions comprising a sunscreen that have the characteristic of being fluid.

The object of the present invention is, precisely, to satisfy all or some of the needs by overcoming the drawbacks mentioned above.

The inventors have observed that it is possible to obtain, from an oily phase structured with at least one gelling polymer, calibrated and spherical oily particles comprising at least one hydrophobic sunscreen that are conformed so as to optimize the deposition on the surface of the skin and to increase the sun protection factor.

According to one of its first aspects, one subject of the present invention is thus calibrated and spherical oily particles comprising at least one hydrophobic sunscreen and comprising at least one oily phase structured with at least one gelling polymer, the said particles having a mean size of less than or equal to 20 μm and the said structured oily phase having a melting point of greater than or equal to 40° C., and their circularity index being between 0.9 and 1.

A subject of the present invention is also calibrated and spherical oily particles comprising at least one hydrophobic sunscreen and at least one oily phase structured with at least one gelling polymer and having a mean size of less than or equal to 20 μm, the gelling polymer being chosen from a semi-crystalline polymer, a polyamide, a silicone polyamide, a polysaccharide monoalkyl ester or polyalkyl ester, a diblock and/or triblock and/or multiblock and/or radial-block copolymer, and mixtures thereof, and their circularity index being between 0.9 and 1.

According to another of its aspects, a subject of the present invention is also calibrated and spherical oily particles comprising at least one hydrophobic sunscreen, obtained from an oily phase structured with at least one gelling polymer, the gelling polymer being of a nature and/or in an amount sufficient to give the said oily phase a viscosity of greater than or equal to 750 Pa·s at a shear of 1 s⁻¹, at 25° C., and their circularity index being between 0.9 and 1.

### DETAILED DESCRIPTION OF EMBODIMENTS

For the purposes of the present invention, the term “sufficient content” is intended to denote the minimum content required to observe the expected effect, i.e., in the case of the gelling polymer, the production of a structured oily phase having the viscosity required to obtain particles in accordance with the present invention.

In the present description hereinbelow, the term “hydrophobic UV-screening agent” means any agent for screening out UV radiation whose solubility in water at 25° C. does not exceed 0.5%.

The calibrated and spherical particles according to the invention may comprise at least one sunscreen.

According to yet another of its aspects, a subject of the present invention is a dispersion in aqueous and/or water-soluble phase comprising particles in accordance with the present invention.

According to yet another of its aspects, a subject of the present invention is also a process for manufacturing a dispersion in accordance with the invention.

According to yet another of its aspects, a subject of the present invention is a cosmetic or dermatological composition comprising at least some particles and/or at least a dispersion in accordance with the invention.

According to yet another of its aspects, a subject of the present invention is also the use of particles and/or of at least one dispersion in accordance with the invention in a photoprotective cosmetic composition.

According to yet another of its aspects, a subject of the present invention is also a non-therapeutic process for making up and/or caring for the skin, comprising at least one step of applying at least one composition in accordance with the invention thereto.

According to another of its aspects, a subject of the present invention is also the use of particles and/or of at least one dispersion in accordance with the invention for the manufacture of a composition for protecting the skin and/or the hair against the harmful effects of UV radiation, in particular sunlight.

It is understood that the compositions in accordance with the invention are intended to be applied to any part of the skin of the human or animal body, particularly any part of the skin, including the lips and the scalp.

The calibration of the particles is advantageous insofar as it ensures that they have homogeneous size and shape distributions and ensures that the sunscreens have increased stability and performance qualities, which may be measured in particular by the improvement in the SPF.

In general, in the context of photoprotection, they make it possible to optimize the deposition on the surface of the skin.

The use of such calibrated and spherical oil particles comprising a sunscreen also makes it possible to introduce additional heat-sensitive active agents into the final composition, whether they are hydrophilic or lipophilic.

Another advantage that emerges from their use is the possibility of obtaining fluid compositions.

In the context of the present invention, the terms “gelled” and “thickened” may be considered as being synonymous with the term “structured” when it is a matter of qualifying the oily particles.

All cited references are incorporated herein by reference in their entireties.

Calibrated and Spherical Particles

The calibrated oily particles comprising at least one sunscreen and comprising at least one structured oil or oily phase in accordance with the invention have a mean size of less than or equal to 20 μm, in particular less than or equal to 15 μm and more particularly less than or equal to 12 μm. Advantageously, the mean size of the particles may range from 100 nm to 20 μm, from 100 nm to 15 μm or even from 150 nm to 12 μm.

For the purposes of the present invention, the term “calibrated” refers to particles having a homogeneous granulometric distribution.

The particles in accordance with the present invention have a mean size of less than 15 μm and allow controlled and advantageous release of the sunscreen onto the skin and/or the hair.

The granulometric distribution qualifies the distribution of the size of the calibrated particles about a mean size. The granulometric distribution may be characterized by a polydispersity index or a coefficient of uniformity. The lower the index or the coefficient, the more uniformly the particle sizes are distributed about a mean size.

Thus, for the submicron calibrated oily particles, in accordance with the invention, i.e. with a mean size of less than 1 micrometer, the granulometric distribution may be characterized by a polydispersity index, noted as PI (dimensionless value characterizing the extent of the granulometric distribution). This index is then advantageously less than or equal to 0.35 and preferably greater than or equal to 0.01.

The size of the submicron calibrated oily particles may be determined, for example, with a laser granulometer functioning on the principle of quasi-elastic light scattering, for instance the B190Plus® machine from Brookhaven Instrument.

For the calibrated oily particles with a mean size of greater than one micrometer, the granulometric distribution may be characterized by a coefficient of uniformity measured using a laser-scattering granulometer, for instance the Master Sizer 2000® machine from Malvern. The calibrated oily particles in accordance with the invention that are greater than one micrometer in size may have a coefficient of uniformity advantageously of less than or equal to 0.45 and preferably greater than or equal to 0.1.

The size of the particles and the homogeneity of the granulometric distribution about a mean size are generally determined by the nature of the process used to obtain them. The processes for obtaining the calibrated and spherical particles in accordance with the invention are described hereinbelow.

The calibrated particles advantageously have a uniform and substantially spherical shape.

The term “substantially spherical” means that the particles are of substantially isotropic shape, i.e. they have a relatively regular morphology.

In the context of the present invention, a parameter relative to the shape factor of the particles is thus defined, for instance the circularity index C, which is defined as the ratio of the total surface area A of the particle to the surface area of the disc having the same perimeter P:
C=4□DA/P
with C between 0.9 and 1.

This measurement may advantageously be performed using a Sysmex FPIA 2100 machine, which is an image analysis granulometer.

Moreover, besides the homogeneity of shape and of size distribution, the calibrated and spherical particles in accordance with the invention are advantageously homogeneous as regards their structure.

Thus, the oily gel of the particle, obtained by structuring at least one oil or oily phase with at least one gelling polymer, advantageously has a uniform structure.

The distribution of the sunscreen is also advantageously uniform throughout the particle.

Oil

The calibrated and spherical oily particles comprising a sunscreen in accordance with the present invention comprise at least one oil or oily phase, especially containing at least one oil that is liquid at room temperature (20-25° C.) and at atmospheric pressure.

For the purposes of the present invention, the term “oily phase” is intended to denote a phase comprising at least one oil. This oil is advantageously a non-volatile oil. For the purposes of the present invention, the term “non-volatile oil” means an oil having a vapour pressure of less than 0.13 Pa.

The oily phase that is suitable for use in particles in accordance with the present invention may comprise, for example, at least one oil chosen from a plant oil, an animal oil, a synthetic oil and a mineral oil, and mixtures thereof.

The oily phase may also comprise at least one volatile oil, which may require particular processing conditions, especially under pressure.

For the purposes of the present invention, the term “volatile oil” means an oil (or non-aqueous medium) that is capable of evaporating on contact with the skin (for example at about 33° C.) in less than one hour, at room temperature and atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

The oils that are suitable for use in the invention may be of natural, plant or mineral origin, or of synthetic origin. They may be of hydrocarbon-based type, for instance triglycerides, esters, alkanes or polyolefins, of silicone type or of fluoro type, and may be modified or non-modified.

For the purposes of the present invention, the term “fluoro oil” means an oil comprising at least one fluorine atom.

For the purposes of the present invention, the term “silicone oil” means an oil comprising at least one silicon atom, and especially at least one Si—O group.

The term “hydrocarbon-based oil” is intended to denote an oil mainly containing hydrogen and carbon atoms and possibly oxygen, nitrogen, sulfur and/or phosphorus atoms.

According to one embodiment, they may be used alone or as a mixture, with each other or with other compounds as defined, for example, hereinbelow.

Advantageously, the oils used for the implementation of the invention are compatible with the gelling polymer used to structure the oily phase.

The non-volatile oils may especially be chosen from non-volatile hydrocarbon-based oils and, where appropriate, fluoro oils and/or silicone oils.

Non-volatile hydrocarbon-based oils that may especially be mentioned include:

hydrocarbon-based oils of animal origin, such as squalane;

hydrocarbon-based oils of plant origin such as phytostearyl esters, such as phytostearyl oleate, phytostearyl isostearate and lauroyl/octyldodecyl/phytostearyl glutamate (Ajinomoto, Eldew PS203); triglycerides consisting of fatty acid esters of glycerol, the fatty acids of which may have chain lengths ranging from C₄ to C₂₄, these chains possibly being linear or branched, and saturated or unsaturated; these oils are especially heptanoic or octanoic triglycerides, wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; shea butter; or alternatively caprylic/capric acid triglycerides, for instance those sold by the company Stearineries Dubois or those sold under the names Miglyol 810®, 812® and 818® by the company Dynamit Nobel, and mixtures thereof;

linear or branched hydrocarbons of mineral or synthetic origin such as petroleum jelly, polydecenes, hydrogenated polyisobutene such as Parleam, and mixtures thereof;

synthetic ethers containing from 10 to 40 carbon atoms;

synthetic esters, for instance oils of formula R₁COOR₂ in which R₁ represents a linear or branched fatty acid residue containing from 1 to 40 carbon atoms and R₂ represents a hydrocarbon-based chain that is especially branched, containing from 1 to 40 carbon atoms provided that R₁+R₂>10;

and mixtures thereof.

The esters may be chosen especially from fatty acid esters, for instance:

cetostearyl octanoate, isopropyl alcohol esters, such as isopropyl myristate, isopropyl palmitate, isopropyl lauroylsarcosinate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, polyethylene glycol diheptanoate, propylene glycol 2-diethylhexanoate and mixtures thereof, hexyllaurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, hydroxylated esters, for instance isostearyl lactate or diisostearyl malate, alkylbenzoate and C₁₂ to C₁₅ alcohol benzoates, and mixtures thereof;

polyol esters and pentaerythritol esters, for instance dipentaerythrityl tetrahydroxystearate/tetraisostearate;

esters of diol dimers and diacid dimers such as Lusplan DD-DA5® and DD-DA7®, and mixtures thereof, sold by the company Nippon Fine Chemical and described in patent application FR 0 302 809 filed on 6 Mar. 2003, the content of which is incorporated into the present patent application by reference;

fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;

higher fatty acids that are liquid such as oleic acid, linoleic acid or linolenic acid, and mixtures thereof; and

dialkyl carbonates, the two alkyl chains possibly being identical or different, such as dicaprylyl carbonate sold under the name Cetiol CC® by Cognis; and

mixtures thereof.

The non-volatile silicone oils that may be used in the composition according to the invention may be non-volatile polydimethylsiloxanes (PDMS), such as simethicone, polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenylmethyl diphenyltrisiloxanes and 2-phenylethyl trimethylsiloxysilicates, and dimethicones or phenyl trimethicones with a viscosity of less than or equal to 100 cSt, and mixtures thereof.

The volatile hydrocarbon-based oils optionally present may especially be chosen from hydrocarbon-based oils containing from 8 to 16 carbon atoms, and especially branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

As volatile oils optionally present, it is also possible to use volatile silicones, for instance volatile linear or cyclic silicone oils, especially those with a viscosity ≦8 centistokes (8×10⁻⁶ m²/s), and especially containing from 2 to 10 silicon atoms and in particular from 2 to 7 silicon atoms, these silicone oils optionally comprising alkyl or alkoxy groups containing from 1 to 10 carbon atoms. As a volatile silicone oil that may be used in the invention, mention may be made especially of dimethicones with a viscosity of 5 and 6 cSt, such as octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethyl-cyclopentane, and mixtures thereof, may also be used.

Advantageously, the oily phase present in the particles in accordance with the present invention may comprise at least one oil chosen especially from squalane, isononyl isononanoate, isopropyl lauroylsarcosinate and octyldodecanol, and mixtures thereof.

The content of oily phase in the particles in accordance with the invention may range from 10% to 99% by weight, in particular from at least 15% to 99% by weight and more particularly from 20% to 99% by weight relative to the total weight of the calibrated particle according to the invention.

According to one embodiment, the oily phase, structured with at least one gelling polymer forming calibrated oily particles in accordance with the invention, has a melting point of greater than or equal to 40° C. and preferably less than or equal to 95° C. According to one preferred embodiment, the same oily phase has a melting point that may range between 50 and 90° C.

The structured oily phase from which the calibrated oily particles in accordance with the invention are derived has a viscosity of greater than 750 Pa·s at a shear of 1 s⁻¹ at 25° C., and preferably less than or equal to 1×10⁶ Pa·s.

The viscosity of the oil or oily phase structured with at least one gelling polymer may be determined using a rheometer (RFS3 from Rheometrics).

The measurements are then taken at 25° C., the temperature being regulated by the Peltier effect.

The geometry is a cone/plate geometry, with a cone 25 mm in diameter and an angle of 2°.

A rate gradient of 1 s⁻¹ is imposed, for a certain equilibrium time, for example 5 minutes. The viscosity is given in Pa·s, at a given temperature and time.

The viscosity is in reality measured at a stage where the particles are not yet formed, i.e. on the oil, gelling agent and hydrophobic sunscreen premix, as emerges on reading the detailed process below.

The choice of the oil(s) included in the formulation of the oily phase and also that of the gelling polymer(s) may be adjusted by a person skilled in the art such that the structured oily phase of the particles in accordance with the invention satisfies the melting point and viscosity criteria described above.

Gelling Polymer

The calibrated and spherical oily particles are advantageously obtained from at least one oily phase structured with at least one gelling polymer chosen especially from a semi-crystalline polymer, a polysaccharide monoalkyl or polyalkyl ester, a polyamide, a silicone polyamide or a diblock, triblock, multiblock and/or radial-block copolymer, and mixtures thereof.

For the purposes of the present invention, the term “polymer” is intended to denote compounds comprising at least two repeating units, preferably at least three repeating units and especially at least 10 repeating units.

The content of gelling polymer(s) in the particles in accordance with the invention may range from 1% to 80% by weight or even from 1% to 60% by weight relative to the total weight of the particle.

Advantageously, the weight ratio between the gelling polymer and the oily phase of the particle according to the invention may range from 0.01 to 4, or even from 0.05 to 2 and in particular from 0.1 to 1.

Semi-Crystalline Polymer

For the purposes of the invention, the term “semi-crystalline polymer” means polymers comprising a crystallizable portion, side chain or block in the skeleton, and an amorphous portion in the skeleton and having a first-order reversible change of phase temperature, in particular of melting (solid-liquid transition). When the crystallizable portion is a block of the polymer skeleton, this crystallizable block has a different chemical nature from that of the amorphous blocks; in this case, the semi-crystalline polymer is a block polymer, for example of the diblock, triblock or multiblock type.

The semi-crystalline polymers that may be used for the implementation of the present invention are solid at room temperature and preferably have a melting point (or gel point) of less than 80° C.

They comprise:

a) a polymer skeleton, and

b) at least one crystallizable organic side chain and/or one crystallizable organic block forming part of the skeleton of the said polymer, the said polymer having a number-average molecular mass of greater than or equal to 2000.

Advantageously, the semi-crystalline polymer(s) of the composition of the invention have a number-average molecular mass Mn of greater than or equal to 2000, for example ranging from 2000 to 800 000 and especially from 3000 to 500 000.

According to one embodiment, the semi-crystalline polymers that may be used in the context of the invention have a melting point m.p. of less than 70° C. and especially of less than 50° C. The semi-crystalline polymer advantageously has a melting point m.p. in the range from 40° C. to less than 80° C. In reality, the semi-crystalline polymer may be a mixture of semi-crystalline polymers. In this case, it is the mixture that has a melting point m.p. within the said range. In other words, the mixture may comprise a semi-crystalline polymer having a melting point outside this range, provided that the mixture itself has a melting point within the said range. The melting point may be measured especially by any known method and in particular with a differential scanning calorimeter (DSC).

According to one embodiment variant, the crystallizable blocks or chains of the semi-crystalline polymers represent at least 30% or even at least 40% of the total weight of each polymer. The semi-crystalline polymers of the invention containing crystallizable blocks may be block or multiblock polymers. They may be obtained by polymerization of monomers containing reactive double bonds (ethylenic bonds) or by polycondensation. When the polymers of the invention are polymers containing crystallizable side chains, they are advantageously in random or statistical form.

The semi-crystalline polymers that may be used in the invention are, for example:

1. block copolymers of polyolefins of controlled crystallization, whose monomers are described in document EP-A-0 951 897,

2. polycondensates, especially of aliphatic or aromatic polyester type or of aliphatic/aromatic copolyester type,

3. homopolymers or copolymers bearing at least one crystallizable side chain and homopolymers or copolymers bearing at least one crystallizable block in the skeleton, for instance those described in patent U.S. Pat. No. 5,156,911,

4. homopolymers or copolymers bearing at least one crystallizable side chain, bearing fluoro group(s) patent application such as those described in WO-A-01/19333,

5. and mixtures thereof.

In the last two cases (3 and 4), the crystallizable side chain(s) or block(s) is (are) hydrophobic.

The crystalline polymers containing crystallizable side chains, or bearing in the skeleton at least one crystallizable block suitable for use in the invention, are, for example, described below.

A) Semi-Crystalline Polymers Containing Crystallizable Side Chains

Mention may be made in particular of the polymers defined in documents U.S. Pat. No. 5,156,911 and WO-A-01/19333. They are homopolymers or copolymers comprising from 50% to 100% by weight of units resulting from the polymerization of one or more monomers bearing (a) crystallizable hydrophobic side chain(s).

These homopolymers or copolymers are of any nature, provided that they meet the conditions mentioned hereinbelow with, in particular, the characteristic of being soluble or dispersible in the oily phase, by heating above their melting point mp (or gel point). They can result:

from the polymerization, especially the free-radical polymerization, of one or more monomers containing (a) reactive or ethylenic double bond(s) with respect to a polymerization, namely a vinyl, (meth)acrylic or allylic group,

from the polycondensation of one or more monomers bearing co-reactive groups (carboxylic acid, sulfonic acid, alcohol, amine or isocyanate), such as, for example, polyesters, polyurethanes, polyethers, polyureas or polyamides.

In general, the crystallizable units (chains or blocks) of semi-crystalline polymers that can be used in the context of the invention are derived from monomer(s) containing (a) crystallizable block(s) or chain(s), used for manufacturing semi-crystalline polymers. These polymers are chosen especially from homopolymers and copolymers resulting from the polymerization of at least one monomer containing (a) crystallizable chain(s) that may be represented by formula X: 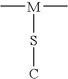
in which M represents an atom of the polymer skeleton, S represents a spacer and C represents a crystallizable group.

The crystallizable chains “—S—C” may be aliphatic or aromatic, and optionally fluorinated or perfluorinated. “S” especially represents a group (CH₂)ₙ or (CH₂CH₂O)ₙ or (CH₂O), which may be linear or branched or cyclic, with n being an integer ranging from 0 to 22. Preferably, “S” is a linear group. Preferably, “S” and “C” are different.

When the crystallizable chains are aliphatic chains, they comprise at least 11 carbon atoms and not more than 40 carbon atoms and better still not more than 24 carbon atoms. They are especially, for example, alkyl chains containing at least 12 carbon atoms, and they can be alkyl chains containing from 14 to 24 carbon atoms C₁₄-C₂₄. They can be hydrocarbon-based alkyl chains (carbon and hydrogen atoms) or fluoroalkyl or perfluoroalkyl chains (carbon atoms, fluorine atoms and optionally hydrogen atoms). When they are fluoroalkyl or perfluoroalkyl chains, they contain at least 11 carbon atoms, at least 6 of which carbon atoms are fluorinated.

As examples of semi-crystalline polymers or copolymers containing (a) crystallizable chain(s), mention may be made of those resulting from the polymerization of at least one monomer with a crystallizable chain chosen from (meth)acrylates of saturated C₁₄-C₂₄ alkyls (C₁₄-C₂₄ means that the alkyl group contains from 14 to 24 carbon atoms); C₁₁-C₁₅ perfluoroalkyl (meth)acrylates (alkyl group containing 11 to 15 carbon atoms); C₁₄ to C₂₄ N-alkyl(meth)acrylamides with or without a fluorine atom; vinyl esters containing C₁₄ to C₂₄ alkyl or perfluoroalkyl chains, with a perfluoroalkyl chain containing at least 6 fluorine atoms; vinyl ethers containing C₁₄ to C₂₄ alkyl or perfluoroalkyl chains, with a perfluoroalkyl chain containing at least 6 fluorine atoms; C₁₄ to C₂₄ α-olefins, for instance octadecene; C₁₄ to C₂₄ para-alkylstyrenes, and mixtures thereof.

For the purposes of the invention, the term “alkyl” means a saturated group especially containing from 8 to 24 carbon atoms (C₈ to C₂₄), except where otherwise mentioned.

When the polymers result from a polycondensation, the hydrocarbon-based and/or fluorinated crystallizable chains as defined above are borne by a monomer that may be a diacid, a diol, a diamine or a diisocyanate.

When the polymers used in the composition of the invention are copolymers, they additionally contain from 0% to 50% of groups Y or Z resulting from copolymerization:
α) with Y which is a polar or non-polar monomer or a mixture of the two:
when Y is a polar monomer, it is either a monomer bearing polyoxyalkylenated groups (especially oxyethylenated and/or oxypropylenated groups), a hydroxyalkyl (meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, or a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.
when Y is a non-polar monomer, it may be an ester of the linear, branched or cyclic alkyl (meth)acrylate type, a vinyl ester, an alkyl vinyl ether, an α-olefin, styrene or styrene substituted with an alkyl group containing from 1 to 10 carbon atoms (C₁ to C₁₀), for instance α-methylstyrene, or a macromonomer of the polyorganosiloxane type containing vinyl unsaturation.
β) with Z which is a polar monomer or a mixture of polar monomers, Z having the same definition as the “polar Y” defined above.

Preferably, the semi-crystalline polymers containing a crystallizable side chain are alkyl (meth)acrylate or alkyl(meth)acrylamide homopolymers with an alkyl group as defined above, and especially of C₁₄-C₂₄, copolymers of these monomers with a hydrophilic monomer preferably of different nature from (meth)acrylic acid, and mixtures thereof. The copolymers can be, for example, alkyl methacrylate copolymers or copolymers of alkylmethacrylamide containing a C₁₄ to C₂₄ alkyl group with N-vinylpyrrolidone or hydroxyethyl (meth)acrylate, and mixtures thereof.

Polymers bearing at least one crystallizable block in the skeleton

This is also a case of polymers that are soluble or dispersible in the oil or oily phase by heating above their melting point mp. These polymers are especially block copolymers consisting of at least 2 blocks of different chemical nature, one of which is crystallizable.

As polymers bearing in the skeleton at least one crystallizable block that are suitable for use in the invention, mention may be made of:

1. the polymers defined in document U.S. Pat. No. 5,156,911;

2. block copolymers of olefin or of cycloolefin containing a crystallizable chain, for instance those derived from the block polymerization of:
cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof,
with ethylene, propylene, 1-butene, 3-methyl-1-butene, 1-hexene, 4-methyl-1-pentene, 1-octene, 1-decene or 1-eicosene, or mixtures thereof. These block copolymers may be in particular (ethylene/norbornene) block copolymers and (ethylene/propylene/ethylidenenorbornene) block terpolymers.

Those resulting from the block copolymerization of at least 2 C₂-C₁₆, better still C₂-C₁₂ α-olefins such as those mentioned above and in particular block bipolymers of ethylene and of 1-octene may also be used.

3. copolymers containing at least one crystallizable block, the rest of the copolymer being amorphous (at room temperature). These copolymers may also contain two crystallizable blocks of different chemical nature. The preferred copolymers are those that simultaneously contain at room temperature a crystallizable block and an amorphous block that are both hydrophobic and lipophilic, sequentially distributed; mention may be made, for example, of polymers containing one of the crystallizable blocks and one of the amorphous blocks below:
block that is crystallizable by nature: a) polyester for instance poly(alkylene terephthalate), b) polyolefin, for instance polyethylenes or polypropylenes.
amorphous and lipophilic block, for instance amorphous polyolefins or copoly(olefin)s such as poly(isobutylene), hydrogenated polybutadiene or hydrogenated poly(isoprene).

As examples of such copolymers containing a crystallizable block and an amorphous block, mention may be made of:

α) poly(ε-caprolactone)-b-poly(butadiene) block copolymers, preferably used hydrogenated, such as those described in the article “Melting behaviour of poly(Σ-caprolactone)-block-polybutadiene copolymers” from S. Nojima, Macromolecules, 32, 3727-3734 (1999),

β) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),

γ) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 25 (1997),

δ) the poly(ethylene)-b-poly(ethylethylene) block copolymers cited in the general article “Crystallization in block copolymers” by I. W. Hamley, Advances in Polymer Science, Vol. 148, 113-137 (1999).

The semi-crystalline polymers that may be used in the context of the invention may be non-crosslinked or partially crosslinked, provided that the degree of crosslinking does not impede their dissolution or dispersion in the liquid oily phase by heating above their melting point. It may then be a case of chemical crosslinking, by reaction with a multifunctional monomer during the polymerization. It may also be a case of physical crosslinking, which may then be due either to the establishment of bonds of hydrogen or dipolar type between groups borne by the polymer, for instance dipolar interactions between carboxylate ionomers, these interactions being in small amount and borne by the polymer skeleton; or due to a phase separation between the crystallizable blocks and the amorphous blocks borne by the polymer.

Preferably, the semi-crystalline polymers that are suitable for the invention are non-crosslinked.

As particular examples of semi-crystalline polymers that may be used in the composition according to the invention, mention may be made of the Intelimer® products from the company Landec described in the brochure “Intelimer® Polymers”. These polymers are in solid form at room temperature (25° C.). They bear crystallizable side chains and contain the monomer as defined in formula X above. Mention may be made especially of “Landec IP22®”, with a melting point m.p. of 56° C., which is a viscous, impermeable, non-tacky product at room temperature.

It is also possible to use the polymer “Structure O” sold by the company National Starch, such as the product described in document U.S. Pat. No. 5,736,125, of m.p. 44° C., and also semi-crystalline polymers containing crystallizable side chains comprising fluoro groups as described in Examples 1, 4, 6, 7 and 8 of document WO-A-01/19333.

It is also possible to use the semi-crystalline polymers obtained by copolymerization of stearyl acrylate and of acrylic acid or of NVP, or by copolymerization of behenyl acrylate and of acrylic acid or NVP, as described in document U.S. Pat. No. 5,519,063 or EP-A-0 550 745.

According to one particular embodiment variant, the semi-crystalline polymers that are suitable for use in the present invention are especially alkyl acrylates, among which mention may be made of the Landec copolymers:

Doresco IPA 13-1®: polystearyl acrylate, m.p. of 49° C. and MW of 145 000;

Doresco IPA 13-3®: polyacrylate/methacrylic acid, m.p. of 65° C. and MW of 114 000;

Doresco IPA 13-4®: polyacrylate/vinylpyrrolidone, m.p. of 44° C. and MW of 387 000;

Doresco IPA 13-5®: polyacrylate/hydroxyethyl methacrylate, m.p. of 47° C. and MW of 397 600;

Doresco IPA 13-6®: polybehenyl acrylate, m.p. of 66° C.

Polyamides

The polyamides that may advantageously be used in the preparation of the particles according to the invention are especially those described in document U.S. Pat. No. 5,783,657 from the company Union Camp.

The polyamides that are suitable for use in the invention especially satisfy the following formula: 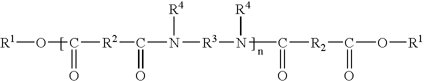
in which:
n is a whole number of amide units such that the number of ester groups represents from 10% to 50% of the total number of ester and amide groups;
R¹ is in each instance, independently, an alkyl or alkenyl group containing at least 4 carbon atoms and especially from 4 to 24 carbon atoms;
R² represents in each instance, independently, a C₄ to C₅₅ hydrocarbon-based group, on condition that at least 50% of the groups R² represent a C₃₀ to C₅₅ hydrocarbon-based group;
R³ represents in each instance, independently, an organic group containing at least 2 carbon atoms, hydrogen atoms and optionally one or more oxygen or nitrogen atoms; and
R⁴ represents in each instance, independently, a hydrogen atom, a C₁ to C₁₀ alkyl group or a direct bond to R³ or to another R⁴ such that the nitrogen atom to which both R³ and R⁴ are attached forms part of a heterocyclic structure defined by R⁴—N—R³, with at least 50% of the groups R⁴ representing a hydrogen atom.

According to one embodiment variant, the ester groups of these polyamides represent from 15% to 40% and at best from 20% to 35% of the total number of ester and amide groups. Furthermore, n advantageously represents an integer ranging from 1 to 10 and better still from 1 to 5.

R¹ is especially a C₁₂ to C₂₂ or even C₁₆ to C₂₂ alkyl group. R² may especially be a C₁₀ to C₄₂ hydrocarbon-based (alkylene) group. In particular, at least 50% and better still at least 75% of the groups R² may be groups containing from 30 to 42 carbon atoms. The other groups R² are C₄ to C₁₉ and in particular C₄ to C₁₂ hydrogen-containing groups. R³ may represent a C₂ to C₃₆ hydrocarbon-based group or a polyoxyalkylene group and R⁴ represents a hydrogen atom. In particular, R³ may represent a C₂ to C₁₂ hydrocarbon-based group. The hydrocarbon-based groups may be linear, cyclic or branched, and saturated or unsaturated groups. Moreover, the alkyl and alkylene groups may be linear or branched, and saturated or unsaturated groups.

As examples of structuring polyamides that may be used in the invention, mention may also be made of polyamide resins resulting from the condensation of an aliphatic dicarboxylic acid and of a diamine (including compounds containing more than two carbonyl groups and two amine groups), the carbonyl and amine groups of adjacent individual units being condensed via an amide bond. These polyamide resins are especially the products sold under the brand name Versamid by the companies General Mills, Inc. and Henkel Corp., under the brand name Onamid, especially Onamid S or C. These resins have a weight-average molecular mass ranging from 6000 to 9000. Documents U.S. Pat. No. 3,645,705 and U.S. Pat. No. 3,148,125 describe these resins. According to one embodiment variant, Versamid 930 or 744 is used.

It is also possible to use the polyamides sold or manufactured by the company Arizona under the references Uni-Rez (2658, 2931, 2970, 2621, 2613, 2624, 2665, 1554, 2623, 2662) and the product sold under the reference Macromelt 6212® by the company Henkel. U.S. Pat. No. 5,500,209 describes polymers of this type.

As examples of structuring polyamides that may be used in the composition according to the invention, mention may also be made of the commercial products sold or manufactured by the company Arizona Chemical under the names Uniclear 80® and Uniclear 100®. They are sold, respectively, in the form of an 80% (active material) gel and a 100% (active material) gel in a mineral oil. They have a softening point of from 88 to 105° C. These commercial products are a mixture of copolymer of a C₃₆ diacid condensed with ethylenediamine, with an average molecular mass of about 6000. The ester end groups result from the esterification of the remaining acid end groups with cetyl or stearyl alcohol or mixtures thereof (also known as cetylstearyl alcohol).

The structuring polyamides advantageously have a softening point of greater than 60° C., which may be up to 190° C. They preferably have a softening point of less than 150° C., ranging from 70 to 130° C. and better still from 80 to 105° C.

The structuring of the oily phase may be obtained by means of one or more polyamides defined above. In general, these polyamides are in the form of mixtures, these mixtures also possibly containing a synthetic product corresponding to a polyamide as defined above with n being 0, i.e. a diester.

The polyamides used in the present invention have, on account of their fatty chain, good solubility in the oily phase and thus lead to compositions that are macroscopically homogeneous, even with a high polymer content.

As examples of polyamides that are suitable for use in the present invention, mention may be made of the copolymer of ethylenediamine/stearyl dimerdilinoleate, sold under the reference Uniclear 100® VG by the company Arizona Chemical.

Silicone Polyamides

The polymers (homopolymers or copolymers) of polyamide type that are suitable for use in the invention have an average molecular mass included in the range from 500 to 500 000 and contain at least one group comprising:

at least one polyorganosiloxane group, comprising from 1 to 1000 organosiloxane units, in the chain of the group or in the form of a graft, and

at least two groups capable of establishing hydrogen interactions, chosen from ester, amide, sulfonamide, carbamate, thiocarabamate, urea, thiourea, oxamido, guanidino and biguanidino groups, and combinations thereof, on condition that at least one of these groups is other than an ester group,

the polymer being solid at room temperature and soluble in the oily phase at a temperature ranging from 25 to 150° C. In particular, the polymer is soluble in the oily phase at a temperature ranging from 41 to 120° C.

The polymers that are suitable for use in the invention, and used as oil-gelling agent, may belong to the following two families:

polyorganosiloxanes comprising at least two groups capable of establishing hydrogen interactions, these two groups being located in the polymer chain, and/or

polyorganosiloxanes comprising at least two groups capable of establishing hydrogen interactions, these two groups being located on grafts or branches.

The polymers comprising two groups capable of establishing hydrogen interactions in the polymer chain may be polymers comprising at least one unit corresponding to the first formula below: 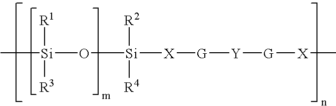
in which:

1. R¹, R², R³ and R⁴, which may be identical or different, represent a group chosen from:

linear, branched or cyclic, saturated or unsaturated C₁ to C₄₀ hydrocarbon-based groups, which may contain in their chain one or more oxygen, sulfur and/or nitrogen atoms, and which may be partially or totally substituted with fluorine atoms,

C₆ to C₁₀ aryl groups optionally substituted with one or more C₁ to C₄ alkyl groups,

polyorganosiloxane chains optionally containing one or more oxygen, sulfur and/or nitrogen atoms;

2. The groups X, which may be identical or different, represent a linear or branched C₁ to C₃₀ alkylenediyl group, which may contain in its chain one or more oxygen and/or nitrogen atoms;

3. Y is a linear or branched alkylene, arylene, cycloalkylene, alkylarylene or arylalkylene, saturated or unsaturated C₁ to C₅₀ divalent group, which may comprise one or more oxygen, sulfur and/or nitrogen atoms, and/or bear as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl groups and C₁ to C₆ aminoalkyl groups, or;

4. Y represents a group corresponding to the formula 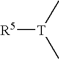
in which:

T represents a linear or branched, saturated or unsaturated C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and

R⁵ represents a linear or branched C₁ to C₅₀ alkyl group, or a polyorganosiloxane chain, possibly comprising one or more ester, amide, urethane, thiocarbamate, urea, thiourea and/or sulfonamide groups which may or may not be linked to another chain of the polymer,

the groups G, which may be identical or different, represent divalent groups chosen from: 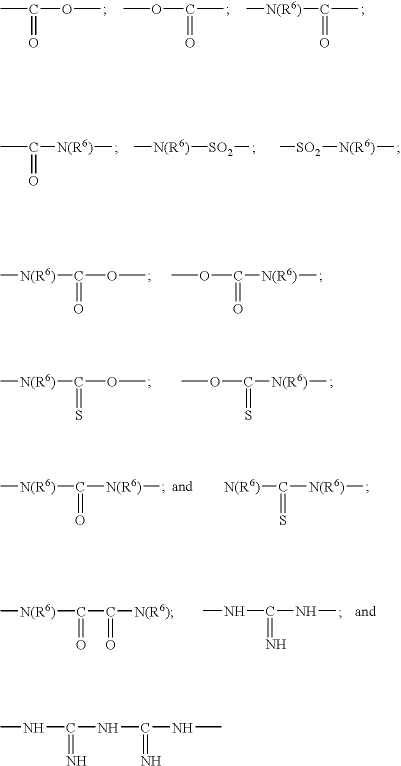
in which R⁶ represents a hydrogen atom or a linear or branched C₁ to C₂₀ alkyl group.

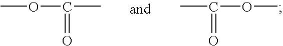

5. n is an integer ranging from 2 to 500 and in particular from 2 to 200, and m is an integer ranging from 1 to 1000, in particular from 1 to 700 and better still from 6 to 200.

According to one embodiment variant, 80% of the groups R¹, R², R³ and R⁴ of the polymer may be chosen especially from methyl, ethyl, phenyl and 3,3,3-trifluoropropyl groups.

According to another embodiment variant, Y may represent various divalent groups, optionally also comprising one or two free valencies to establish bonds with other units of the polymer or copolymer. Y may especially represent a group chosen from:

A) linear C₁ to C₂₀ and especially C₁ to C₁₀ alkylene groups;

B) C₃₀ to C₅₆ branched alkylene groups possibly comprising rings and unconjugated unsaturations;

C) C₅-C₆ cycloalkylene groups;

D) phenylene groups optionally substituted with one or more C₁ to C₄₀ alkyl groups;

E) C₁ to C₂₀ alkylene groups, comprising from 1 to 5 amide groups;

F) C¹ to C₂₀ alkylene groups, comprising one or more substituents, chosen from hydroxyl, C₃ to C₈ cycloalkane, C¹ to C₃ hydroxyalkyl and C¹ to C₆ alkylamine groups;

G) polyorganosiloxane chains of formula: 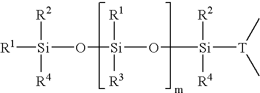
in which R¹, R², R³ and R⁴, T and m are as defined above; and

H) polyorganosiloxane chains of formula: 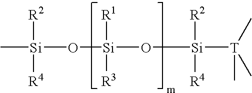
The polyorganosiloxanes of the second family may be polymers comprising at least one unit corresponding to the second formula below: 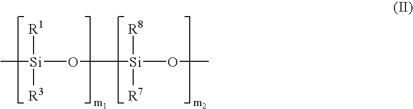
in which:

R¹ and R³, which may be identical or different, are as defined above for the preceding formula;

R⁷ represents a group as defined above for R¹ and R³, or represents the group of formula —X-G-R⁹ in which X and G are as defined above for the preceding formula and R⁹ represents a hydrogen atom or a linear, branched or cyclic, saturated or unsaturated C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups;

R⁸ represents a group of formula —X-G-R⁹ in which X, G and R⁹ are as defined above;

m₁ is an integer ranging from 1 to 998; and

m₂ is an integer ranging from 2 to 500.

According to the invention, the silicone polyamide used as gelling agent may be a homopolymer, i.e. a polymer comprising several identical units, in particular units according to the formulae defined above.

According to the invention, it is also possible to use a silicone polyamide consisting of a copolymer comprising several different units according to the first formula above, i.e. a polymer in which at least one of the groups R¹, R², R³, R⁴, X, G, Y, m and n is different in one of the units. The copolymer may also be formed from several units according to the second formula above, in which at least one of the groups R¹, R³, R⁷, R⁸, m₁ and m₂ is different in at least one of the units.

It is also possible to use a copolymer comprising at least one unit according to the first formula and at least one unit according to the second formula, the units according to the first formula and the units according to the second formula possibly being identical to or different from each other.

According to one variant of the invention, it is also possible to use a silicone polyamide of copolymer type also comprising at least one hydrocarbon-based unit comprising two groups capable of establishing hydrogen interactions chosen from ester, amide, sulfonamide, carbamate, thiocarbamate, urea and thiourea groups, and combinations thereof. These copolymers may be block copolymers, sequenced copolymers or grafted copolymers.

According to one embodiment variant, the groups capable of establishing hydrogen interactions are amide groups of formulae —C(O)NH— and —HN—C(O)—. In this case, the gelling agent may be, for example, a polymer comprising at least one unit according to the third or fourth formula below: 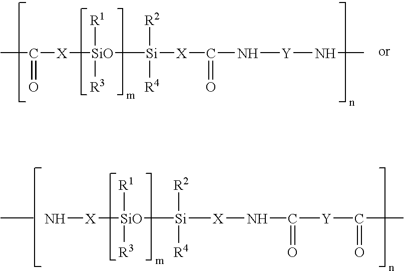
in which R¹, R², R³, R⁴, X, Y, m and n are as defined above.

Such a unit may be obtained:

either via a condensation reaction between a silicone containing α,ω-carboxylic acid end groups and one or more diamines, according to the following reaction scheme: 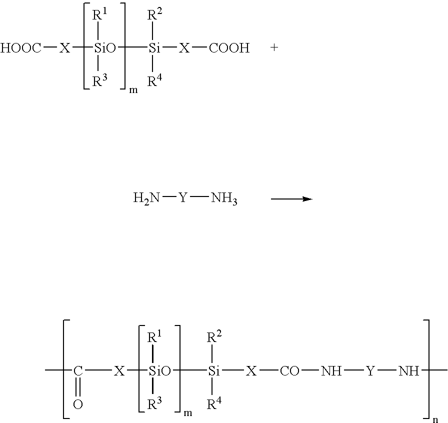
or via reaction of two α-unsaturated carboxylic acid molecules with a diamine according to the following reaction scheme: CH₂═CH—X¹—COOH+H₂N—Y—NH₂->CH₂═CH—X¹—CO—NH—Y—NH—CO—X¹—CH═CH₂ followed by addition of a siloxane to the ethylenic unsaturations, according to the following scheme: CH₂═CH—X¹—CO—NH—Y—NH—CO—X¹—CH═CH₂ 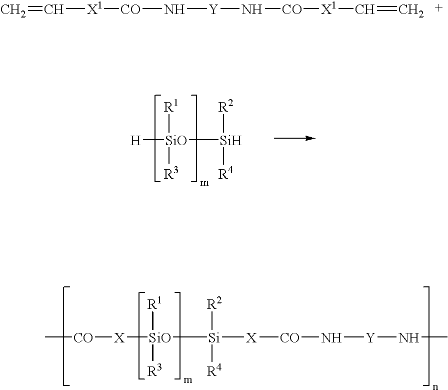
in which X¹—(CH₂)₂— corresponds to X defined above and Y, R¹, R², R³, R⁴ and m are as defined above;

or via reaction with a silicone containing α,ω-NH₂ end groups and of a diacid of formula HOOC—Y—COOH according to the following reaction scheme:

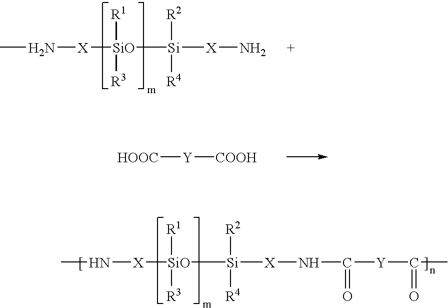

In the polyamides according to the third and fourth formulae presented above:

m is especially in the range from 1 to 700, or even from 15 to 500 and better still from 15 to 45, and

n is in particular in the range from 1 to 500, especially from 1 to 100 and better still from 4 to 25,

X is especially a linear or branched alkylene chain containing from 1 to 30 carbon atoms and in particular 3 to 10 carbon atoms, and

Y is especially a linear or branched alkylene chain or a chain that may comprise rings and/or unsaturations, containing from 1 to 40 carbon atoms, in particular from 1 to 20 carbon atoms and better still from 2 to 6 carbon atoms, in particular 6 carbon atoms.

In the third and fourth formulae presented above, the alkylene group representing X or Y may optionally contain in its alkylene part at least one of the following elements:

1) 1 to 5 amide, urea or carbamate groups,

2) a C₅ or C₆ cycloalkyl group, and

3) a phenylene group optionally substituted with 1 to 3 identical or different C₁ to C₃ alkyl groups.

In the third and fourth formulae presented above, the alkylene groups may also be substituted with at least one element chosen from the group consisting of:

a hydroxyl group,

a C₃ to C₈ cycloalkyl group,

one to three C₁ to C₄₀ alkyl groups,

a phenyl group optionally substituted with one to three C₁ to C₃ alkyl groups,

a C₁ to C₃ hydroxyalkyl group, and

a C₁ to C₆ aminoalkyl group. In the third and fourth formulae presented above, Y may also represent: 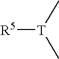
in which R⁵ represents a polyorganosiloxane chain, and T represents a group of formula: 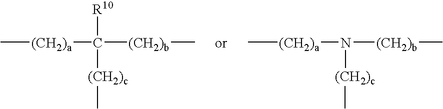
in which a, b and c are, independently, integers ranging from 1 to 10, and R¹⁰ is a hydrogen atom or a group such as those defined for R¹, R², R¹ and R⁴.

In the third and fourth formulae presented above, R¹, R², R³ and R⁴ especially represent, independently, a linear or branched C₁ to C₄₀ alkyl group, in particular a CH₃, C₂H₅, n-C₃H₇ or isopropyl group, a polyorganosiloxane chain or a phenyl group optionally substituted with one to three methyl or ethyl groups.

As has been seen previously, the polymer may also comprise identical or different units according to the third or fourth formula presented above.

Thus, the polymer may be a silicone polyamide containing several units according to the third or fourth formula presented above, of different lengths, or a polyamide corresponding to the fifth formula below: 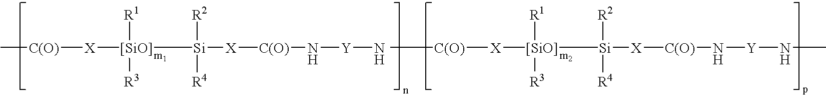
in which X, Y, n, R¹ to R⁴ have the meanings given above, m₁ and m₂, which are different, are chosen in the range from 1 to 1000, and p is an integer ranging from 2 to 300.

In this formula, the units may be structured to form either a block copolymer or a random copolymer or an alternating copolymer. In this copolymer, the units may be not only of different lengths but also of different chemical structures, for example having different groups Y. In this case, the copolymer may correspond to the sixth formula: 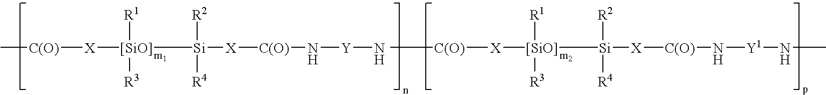
in which R¹ to R⁴, X, Y, m₁, m₂, n and p have the meanings given above and Y¹ is different from Y, but is chosen from the groups defined for Y. As previously, the various units may be structured to form either a block copolymer or a random copolymer or an alternating copolymer.

According to one embodiment of the invention, the gelling silicone polyamide may also consist of a grafted copolymer. Thus, the polyamide containing silicone units may be grafted and optionally crosslinked with silicone chains containing amide groups. Such polymers may be synthesized with trifunctional amines.

In this case, the copolymer may comprise at least one unit according to the seventh formula below: 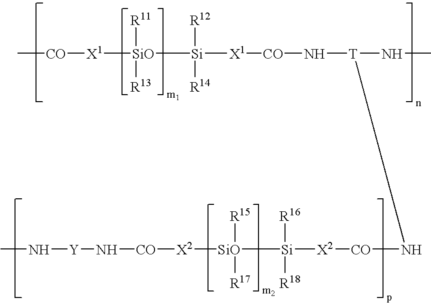
in which X¹ and X², which may be identical or different, have the meaning given for X in the first formula above, n is as defined in the first formula above, Y and T are as defined in the first formula above, R¹¹ to R¹⁸ are groups chosen from the same group as the groups R¹ to R⁴, m₁ and m₂ are numbers in the range from 1 to 1000, and p is an integer ranging from 2 to 500.

In the seventh formula presented above, in particular:

p is in the range from 1 to 25 and better still from 1 to 7,

R¹¹ to R¹⁸ are methyl groups,

T corresponds to one of the following formulae: 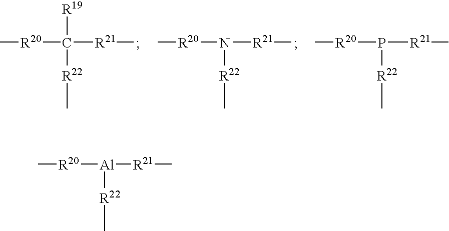
in which R¹⁹ is a hydrogen atom or a group chosen from the groups defined for R¹ to R⁴, and R²⁰, R²¹ and R²² are, independently, linear or branched alkylene groups,

T preferably corresponds in particular to the formula: 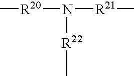
with, especially, R²⁰, R²¹ and R²² representing —CH₂—CH₂—,

m₁ and m₂ are in the range from 15 to 500 or even from 15 to 45,

—X¹ and X² represent —(CH₂)₁₀—, and

Y represents —CH₂—.

These polyamides containing a grafted silicone unit according to the seventh formula presented above may be copolymerized with silicone polyamides according to the second formula to form block copolymers, alternating copolymers or random copolymers. The weight percentage of grafted silicone units according to the seventh formula in the copolymer may range from 0.5% to 30% by weight.

According to one embodiment, the siloxane units may be in the main chain or skeleton of the polymer, but they may also be present in grafted chains or side chains. In the main chain, the siloxane units may be in the form of segments as described above. In the side chains or grafted chains, the siloxane units may appear individually or in segments.

According to one embodiment of the invention, the siloxane-based polyamides may especially be:

polyamides according to the third formula presented above in which m is from 15 to 50;

mixtures of two or more polyamides in which at least one polyamide has a value of m in the range from 15 to 50 and at least one polyamide has a value of m in the range from 30 to 50; polymers according to the fifth formula described above with m₁ chosen in the range from 15 to 50 and m₂ chosen in the range from 30 to 500 with the part corresponding to m₁ representing 1% to 99% by weight relative to the total weight of the polyamide and the part corresponding to m₂ representing 1% to 99% by weight relative to the total weight of the polyamide;

polyamide blends according to the third formula described above, combining:

1. 80% to 99% by weight of a polyamide in which n is equal to 2 to 10 and in particular 3 to 6, and

2. 1% to 20% of a polyamide in which n is in the range from 5 to 500 and in particular from 6 to 100;

polyamides corresponding to the sixth formula presented above in which at least one of the groups Y and Y′ contains at least one hydroxyl substituent;

polyamides according to the third formula synthesized with at least part of an activated diacid (diacid chloride, dianhydride or diester) instead of the diacid;

polyamides according to the third formula in which X represents —(CH₂)₃— or —(CH₂)₁—; and

polyamides according to the third formula in which the polyamides end with a monofunctional chain chosen from the group consisting of monofunctional amines, monofunctional acids, monofunctional alcohols, including fatty acids, fatty alcohols and fatty amines, for instance octylamine, octanol, stearic acid and stearyl alcohol.

According to one embodiment of the invention, the ends of the polymer chains may end with:

a C₁ to C₅₀ alkyl ester group by introducing during the synthesis a C¹ to C₅₀ monoalcohol,

a C₁ to C₅₀ alkylamide group by taking as stopper a monoacid if the silicone contains α,ω-diamino, or a monoamine if the silicone contains α,ω-dicarboxylic acid.

According to one embodiment variant of the invention, it is possible to use a copolymer of silicone polyamide and of hydrocarbon-based polyamide, i.e. a copolymer comprising units according to the third or fourth formula and hydrocarbon-based polyamide units. In this case, the polyamide-silicone units may be located at the ends of the hydrocarbon-based polyamide.

Polyamide-based gelling agents containing silicones may be produced by silyl amidation of polyamides based on fatty acid dimer. This approach involves the reaction of free acid sites existing on a polyamide as end sites, with oligosiloxane-monoamines and/or oligosiloxane-diamines (amidation reaction), or alternatively with oligosiloxane alcohols or oligosiloxane diols (esterification reaction). The esterification reaction requires the presence of acid catalysts, as is known in the art. It is desirable for the polyamide containing free acid sites, used for the amidation or esterification reaction, to have a relatively high number of acid end groups (for example polyamides with high acid numbers, for example from 15 to 20).

For the amidation of the free acid sites of the hydrocarbon-based polyamides, siloxane diamines with 1 to 300, more particularly 2 to 50 and better still 2, 6, 9.5, 12, 13.5, 23 or 31 siloxane groups may be used for the reaction with hydrocarbon-based polyamides based on fatty acid dimers. Siloxane diamines containing 13.5 siloxane groups are preferred, and the best results are obtained with the siloxane-diamine containing 13.5 siloxane groups and polyamides with high numbers of carboxylic acid end groups.

The reactions may be performed in xylene to extract the water produced from the solution by azeotropic distillation, or at higher temperatures (about 180 to 200° C.) without solvent. Typically, the amidation efficacy and the reaction rates decrease when the siloxane diamine is longer, i.e. when the number of siloxane groups is higher. Free amine sites may be blocked after the initial amidation reaction of the diaminosiloxanes by reacting them either with an acidic siloxane or an organic acid such as benzoic acid.

For the esterification of the free acid sites on the polyamides, this may be performed in boiling xylene with about 1% by weight, relative to the total weight of the reagents, of para-toluenesulfonic acid as catalyst.

These reactions performed on the carboxylic acid end groups of the polyamide lead to the incorporation of silicone units only at the ends of the polymer chain.

As an example of a gelling polymer of silicone polyamide type that is suitable for use in the invention, mention may be made of the polyamide/polydimethylsiloxane block copolymer sold, for example, under the reference DC2-8178 Gellant by the company Dow Corning (INCI name Nylon-611/dimethicone copolymer (and) PPG-3 myristyl ether).

Polysaccharide Monoalkyl or Polyalkyl Esters

Among the saccharide or polysaccharide monoalkyl or polyalkyl esters that are suitable for use in the invention, mention may be made of dextrin or inulin alkyl or polyalkyl esters.

It may especially be a dextrin mono- or polyester of at least one fatty acid corresponding especially to the following formula: 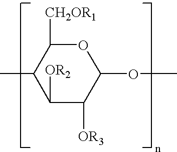
in which:

n is an integer ranging from 3 to 200, especially ranging from 20 to 150 and in particular ranging from 25 to 50,

the radicals R₁, R₂ and R₃, which may be identical or different, are chosen from hydrogen and an acyl group (R—CO—) in which the radical R is a linear or branched, saturated or unsaturated hydrocarbon-based group containing from 7 to 29, in particular from 7 to 21, especially from 11 to 19, more particularly from 13 to 17, or even 15, carbon atoms, with the proviso that at least one of the said radicals R₁, R₂ or R₃ is other than hydrogen.

In particular, R₁, R₂ and R₃ may represent hydrogen or an acyl group (R—CO—) in which R is a hydrocarbon-based radical as defined above, with the proviso that at least two of the said radicals R₁, R₂ or R₃ are identical and other than hydrogen.

The radicals R₁, R₂ and R₃ may all contain an acyl group (R—CO), which is identical or different and especially identical.

In particular, n mentioned above advantageously ranges from 25 to 50 and is especially equal to 38 in the general formula of the saccharide ester that may be used in the present invention.

When the radicals R₁, R₂ and/or R₃, which may be identical or different, contain an acyl group (R—CO), these radicals may be chosen especially from caprylic, capric, lauric, myristic, palmitic, stearic, arachic, behenic, isobutyric, isovaleric, 2-ethylbutyric, ethylmethylacetic, isoheptanoic, 2-ethylhexanoic, isononanoic, isodecanoic, isotridecanoic, isomyristic, isopalmitic, isostearic, isoarachic, isohexanoic, decenoic, dodecenoic, tetradecenoic, myristoleic, hexadecenoic, palmitoleic, oleic, elaidic, asclepinic, gondoleic, eicosenoic, sorbic, linoleic, linolenic, punicic, stearidonic, arachidonic and stearolic radicals, and mixtures thereof.

Preferably, at least one dextrin palmitate is used as dextrin ester of fatty acid(s). This ester may be used alone or as a mixture with other esters.

Advantageously, the dextrin ester of fatty acid has a degree of substitution of less than or equal to 2.5, especially ranging from 1.5 to 2.5, and preferably from 2 to 2.5, on the basis of one glucose unit. The weight-average molecular weight of the dextrin ester may in particular be from 10 000 to 150 000, especially from 12 000 to 100 000, or even from 15 000 to 80 000.

Dextrin esters, in particular dextrin palmitates, are commercially available under the name Rheopearl TL or Rheopearl KL by the company Chiba Flour.

Diblock, triblock, multiblock, radial-block or star copolymers

The block polymers that are suitable for use in the invention are especially those described in patents U.S. Pat. No. 5,756,082 and EP 0 497 144, and also in patent application WO 98/42298, which are incorporated into the present patent application by reference.

Also, the block polymers that may be used in the present invention may be chosen from:

block (diblock or triblock) copolymers such as the polystyrene silicones or the polyethylene silicones described in patents U.S. Pat. No. 6,225,390, U.S. Pat. No. 6,160,054, U.S. Pat. No. 6,174,968 and U.S. Pat. No. 6,225,390,

block or grafted copolymers comprising a silicone block and another block or graft of polyvinyl or polymethacrylic type, such as those described in patents U.S. Pat. No. 5,468,477 and U.S. Pat. No. 5,725,882,

polymers or copolymers resulting from the polymerization or copolymerization of an ethylenic monomer, comprising one or more optionally conjugated ethylenic bonds (or dienes),

polymers or copolymers resulting from the polymerization or copolymerization of an ethylenic monomer, especially a copolymer of vinyl, acrylic or methacrylic type, which may be a block copolymer, such as a diblock, triblock or even multiblock copolymer or a radial or star copolymer.

The gelling agent of ethylenic type may comprise, for example, a styrene block, an alkylstyrene block, an ethylene/butylene block, an ethylene/propylene block, a butadiene block, and isoprene block, an acrylate block or a methacrylate block, or a combination of these blocks.

According to one embodiment, the diblock, triblock, multiblock and/or radial or star copolymers may comprise at least two thermodynamically incompatible segments.

A diblock copolymer is usually defined as being of A-B type or as a block in which a hard segment (A) is followed by a soft segment (B).

A triblock copolymer is usually defined as being of A-B-A type or as a ratio of a hard segment, a soft segment and a hard segment.

A multiblock, radial or star copolymer may comprise any type of combination of hard segments and soft segments, with the proviso that the characteristics of the hard segments and of the soft segments are conserved.

An example of hard segments of block copolymers that may be mentioned is styrene, and examples of soft segments of block copolymers that may be mentioned include ethylene, propylene and butylene, and a combination thereof.

The triblock copolymers, and especially those of polystyrene/polyisoprene or polystyrene/polybutadiene type, which is suitable for use in the invention may be those sold under the reference Luvitol HSB by the company BASF. Mention may also be made of triblock copolymers of polystyrene/copoly(ethylene-propylene) or polystyrene/copoly(ethylene-butylene) type, such as those sold under the reference Kraton by the company Shell Chemical Co., or under the reference Gelled Permethyl 99 A by the company Penreco.

As a further example of block copolymers that may be suitable for use in the present invention, mention may also be made of the block copolymers sold under the reference Versagel by the company Penreco, those sold under the reference Kraton by the company Shell and those sold under the reference Gel Base by the company Brooks Industries.

Among the oily-phase-gelling polymers that are suitable for use in the invention, mention may be made especially of the copolymer of ethylenediamine/stearyl dimerdilinoleate and the semi-crystalline polymer of poly-C₁₀₋₃₀ alkyl acrylate type, and mixtures thereof.

According to one embodiment variant of the present invention, the structured calibrated oily particles may especially contain isopropyl lauroylsarcosinate as oil, and an ethylenediamine dimerdilinoleate/stearyl copolymer as gelling polymer.

According to another embodiment variant, the structured calibrated oily particles may comprise squalane as oil and a semi-crystalline polymer of poly-C₁₀₋₃₀ alkylacrylate type as gelling polymer.

According to yet another embodiment, the structured calibrated oily particles may comprise a mixture of isopropyl laurylsarcosinate and isononyl isononanoate as oily phase and the ethylenediamine dimerdilinoleate/stearyl copolymer as gelling polymer.

According to yet another embodiment, the structured calibrated oily particles in accordance with the present invention may comprise a mixture of isononyl isononanoate and octyldodecanol as oily phase and the ethylenediamine dimerdilinoleate/stearyl copolymer as gelling polymer.

In addition, the particles may comprise another gelling agent of alkylglutamic acid amide derivative type, for example the laurylglutamic acid dibutylamide sold by the company Ajinomoto under the name “Gelling Agent GP-1”.

Sunscreens

The particles in accordance with the invention comprise at least one hydrophobic sunscreen, which may be chosen from a hydrophobic organic photoprotective agent and an inorganic photoprotective agent that is active in the UVA and/or UVB range (absorbers), and mixtures thereof.

The hydrophobic organic screening agents are chosen especially from anthranilates; cinnamic derivatives; dibenzoylmethane derivatives; salicylic derivatives; camphor derivatives; triazine derivatives such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β-diphenylacrylate derivatives; benzotriazole derivatives; benzalmalonate derivatives; benzimidazole derivatives; imidazolines; bis-benzoazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; screening polymers and screening silicones such as those described especially in patent application WO 93/04665; dimers derived from α-alkylstyrene, such as those described in patent application DE 198 55 649; 4,4-diarylbutadienes such as those described in patent applications EP 0 967 200, DE 197 46 654, DE 197 55 649, EP-A-1 008 586, EP 1 133 980 and EP 133 981, and mixtures thereof.

As examples of hydrophobic organic screening agents, mention may be made of those denoted hereinbelow under their INCI name:

para-Aminobenzoic acid derivatives:
Ethyl PABA,
Ethyl dihydroxypropyl PABA,
Ethylhexyl dimethyl PABA sold in particular under the name “Escalol 507” by ISP.

Salicylic derivatives:
Homosalate sold under the name “Eusolex HMS” by Rona/EM Industries,
Ethylhexyl salicylate sold under the name “Neo Heliopan OS” by Haarmann & Reimer.

Dibenzoylmethane derivatives:
Butylmethoxydibenzoylmethane sold in particular under the trade name “Parsol 1789” by Hoffmann LaRoche,
Isopropyldibenzoylmethane.

Cinnamic derivatives:
Ethylhexyl methoxycinnamate sold in particular under the trade name “Parsol MCX” by Hoffmann LaRoche,
Isopropyl methoxycinnamate,
Isoamyl methoxycinnamate sold under the trade name “Neo Heliopan E 1000” by Haarmann & Reimer,
Diisopropyl methylcinnamate.

β,β-Diphenylacrylate Derivatives:
Octocrylene sold in particular under the trade name “Uvinul N539” by BASF,
Etocrylene sold in particular under the trade name “Uvinul N35” by BASF.

Benzophenone derivatives:
Benzophenone-1 sold under the trade name “Uvinul 400” by BASF,
Benzophenone-2 sold under the trade name “Uvinul D50” by BASF,
Benzophenone-3 or Oxybenzone sold under the trade name “Uvinul M40” by BASF,
Benzophenone-6 sold under the trade name “Helisorb 11” by Norquay,
Benzophenone-8 sold under the trade name “Spectra-Sorb UV-24” by American Cyanamid,
Benzophenone-10,
Benzophenone-11,
Benzophenone-12,
Diethylaminohydroxybenzoylhexyl benzoate sold under the trade name “Uvinul A Plus” by BASF.

Benzylidenecamphor derivatives:
3-Benzylidenecamphor manufactured under the name “Mexoryl SD” by Chimex,
Methylbenzylidenecamphor sold under the name “Eusolex 6300” by Merck,
Polyacrylamidomethylbenzylidenecamphor manufactured under the name “Mexoryl SW” by Chimex.

Triazine derivatives:
bis(ethylhexyloxyphenol)methoxyphenyltriazine sold under the trade name “Tinosorb S” by Ciba Geigy,
Ethylhexyltriazone sold in particular under the trade name “Uvinul T150” by BASF,
Diethylhexylbutamidotriazone sold under the trade name “Uvasorb HEB” by Sigma 3V,
2,4,6-tris(dineopentyl 4′-aminobenzalmalonate)-s-triazine,
2,4,6-tris(diisobutyl 4′-aminobenzalmalonate)-s-triazine.

Phenylbenzotriazole derivatives:
Drometrizole trisiloxane sold under the name “Silatrizole” by Rhodia Chimie, methylenebis(benzotriazolyl)tetramethylbutylphenol sold in solid form under the trade name “MIXXIM BB/100” by Fairmount Chemical, or in micronized form as an aqueous dispersion under the trade name “Tinosorb M” by Ciba Specialty Chemicals.

Anthranilic derivatives:
Menthyl anthranilate sold under the trade name “Neo Heliopan MA” by Haarmann & Reimer.

Imidazoline derivatives:
Ethylhexyldimethoxybenzylidenedioxoimidazoline propionate.

Benzalmalonate derivatives:
Polyorganosiloxane containing benzalmalonate functions, for instance Polysilicone-15, sold under the trade name “Parsol SLX” by Hoffmann LaRoche.

4,4-Diarylbutadiene derivatives:
1,1-Dicarboxy(2,2′-dimethylpropyl)-4,4-diphenylbutadiene,

Benzoxazole derivatives:
2,4-bis[5-1 (dimethylpropyl)benzoxazol-2-yl(4-phenyl)imino]-6-(2-ethylhexyl)imino-1,3,5-triazine sold under the name Uvasorb K2A by Sigma 3V
and mixtures thereof.

The preferred hydrophobic organic UV-screening agents are chosen from:
Ethylhexyl salicylate,
Homosalate,
Ethylhexyl methoxycinnamate,
Butylmethoxydibenzoylmethane,
Octocrylene,
Benzophenone-3,
n-Hexyl 2-(4-diethylamino-2-hydroxybenzoyl)benzoate,
4-Methylbenzylidenecamphor,
Bis(ethylhexyloxyphenol)methoxyphenyltriazine,
Ethylhexyltriazone,
Diethylhexylbutamidotriazone,
Methylenebis(benzotriazolyl)tetramethylbutylphenol,
Drometrizole trisiloxane,
Polysilicone-15,
1,1-Dicarboxy(2,2′-dimethylpropyl)-4,4-diphenylbutadiene,
2,4-Bis[5-1 (dimethylpropyl)benzoxazol-2-yl(4-phenyl)imino]-6-(2-ethylhexyl)imino-1,3,5-triazine,
and mixtures thereof.

The particles in accordance with the invention may comprise mineral protective agents. However, they are preferably present in small amounts, as is detailed hereinbelow.

The mineral photoprotective agents are chosen from pigments and even more preferentially nanopigments (mean size of the primary particles: generally between 5 nm and 100 nm and preferably between 10 nm and 50 nm) of treated or untreated metal oxides such as, for example, nanopigments of titanium oxide (amorphous or crystallized in rutile and/or anatase form), or of iron oxide, zinc oxide, zirconium oxide or cerium oxide.

The treated nanopigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina or glycerol.

The treated nanopigments may more particularly be titanium oxides treated with:
silica and alumina, such as the products “Microtitanium Dioxide MT 500 SA” and “Microtitanium dioxide MT 100 SA” from the company Tayca, and the products “Tioveil Fin”, “Tioveil OP”, “Tioveil MOTG” and “Tioveil IPM” from the company Tioxide,
alumina and aluminium stearate, such as the product “Microtitanium Dioxide MT 100 T” from the company Tayca,
alumina and aluminium laurate, such as the product “Microtitanium Dioxide MT 100 S” from the company Tayca,
iron oxides and iron stearate, such as the product “Microtitanium Dioxide MT 100 F” from the company Tayca,
silica, alumina and silicone, such as the products “Microtitanium Dioxide MT 100 SAS”, “Microtitanium Dioxide MT 600 SAS” and “Microtitanium Dioxide MT 500 SAS” from the company Tayca,
sodium hexametaphosphate, such as the product “Microtitanium Dioxide MT 150 W” from the company Tayca,
octyltrimethoxysilane, such as the product “T-805” from the company Degussa,
alumina and stearic acid, such as the product “UVT-M160” from the company Kemira,
alumina and glycerol, such as the product “UVT-M212” from the company Kemira,
alumina and silicone, such as the product “UVT-M262” from the company Kemira.

Other titanium oxide nanopigments treated with a silicone are preferably TiO₂ treated with octyltrimethylsilane and for which the mean size of the elementary particles is between 25 and 40 nm, such as the product sold under the trade name “T 805” by the company Degussa Silices, TiO₂ treated with a polydimethylsiloxane and for which the mean size of the elementary particles is 21 nm, such as the product sold under the trade name “70250 Cardre UF TiO2SI3” by the company Cardre, anatase/rutile TiO₂ treated with a polydimethylhydrogenosiloxane and for which the mean size of the elementary particles is 25 nm, such as the product sold under the trade name “Microtitanium Dioxide USP Grade Hydrophobic” by the company Color Techniques.

The uncoated titanium oxide nanopigments are sold, for example, by the company Tayca under the trade names “Microtitanium Dioxide MT 500 B” or “Microtitanium Dioxide MT 600 B”, by the company Degussa under the name “P 25”, by the company Wackher under the name “Oxyde de titane transparent PW”, by the company Miyoshi Kasei under the name “UFTR”, by the company Tomen under the name “ITS” and by the company Tioxide under the name “Tioveil AQ”.

The uncoated zinc oxide nanopigments are, for example:
those sold under the name “Z-Cote” by the company Sunsmart;
those sold under the name “Nanox” by the company Elementis;
those sold under the name “Nanogard WCD 2025” by the company Nanophase Technologies.

The coated zinc oxide nanopigments are, for example:

those sold under the name “Zinc Oxide CS-5” by the company Toshibi (ZnO coated with polymethylhydrogenosiloxane);
those sold under the name “Nanogard Zinc Oxide FN” by the company Nanophase Technologies (as a 40% dispersion in Finsolv TN, C₁₂-C₁₅ alkyl benzoate);
those sold under the name “Daitopersion ZN-30” and “Daitopersion ZN-50” by the company Daito (dispersions in cyclopolymethylsiloxane/oxyethylenated polydimethylsiloxane, containing 30% or 50% of nanozinc oxides coated with silica and polymethylhydrogenosiloxane);
those sold under the name “NFD Ultrafine ZNO” by the company Daikin (ZnO coated with perfluoroalkyl phosphate and copolymer based on perfluoroalkylethyl as a dispersion in cyclopentasiloxane);
those sold under the name “SPD-ZI” by the company Shin-Etsu (ZnO coated with silicone-grafted acrylic polymer, dispersed in cyclodimethylsiloxane);
those sold under the name “Escalol Z100” by the company ISP (alumina-treated ZnO dispersed in an ethylhexyl methoxycinnamate/PVP-hexadecene copolymer/methicone mixture);
those sold under the name “Fuji ZnO-SMS-10” by the company Fuji Pigment (ZnO coated with silica and polymethylsilsesquioxane);
those sold under the name “Nanox Gel TN” by the company Elementis (ZnO dispersed at a concentration of 55% in C₁₂-C₁₅ alkyl benzoate with hydroxystearic acid polycondensate).

The uncoated cerium oxide nanopigments are sold under the name “Colloidal Cerium Oxide” by the company Rhˆne-Poulenc.

The uncoated iron oxide nanopigments are sold, for example, by the company Arnaud under the names “Nanogard WCD 2002 (FE 45B)” and “Nanogard Iron FE 45 BL AQ”, “Nanogard FE 45R AQ”, “Nanogard WCD 2006 (FE 45R)” or by the company Mitsubishi under the name “TY-220”.

The coated iron oxide nanopigments are sold, for example, by the company Arnaud under the names “Nanogard WCD 2008 (FE 45B FN)”, “Nanogard WCD 2009 (FE 45B 556)”, “Nanogard FE 45 BL 345” and “Nanogard FE 45 BL” or by the company BASF under the name “Transparent Iron Oxide”.

Mention may also be made of mixtures of metal oxides, especially of titanium dioxide and of cerium dioxide, including the silica-coated equal-weight mixture of titanium dioxide and of cerium dioxide, sold by the company Ikeda under the name “Sunveil A”, and also the alumina, silica and silicone-coated mixture of titanium dioxide and of zinc dioxide, such as the product “M 261” sold by the company Kemira, or the alumina, silica and glycerol-coated mixture of titanium dioxide and of zinc dioxide, such as the product “M 211” sold by the company Kemira.

The nanopigments may be introduced into the particles according to the invention in unmodified form or in the form of pigmentary paste, i.e. as a mixture with a dispersant, as described, for example, in document GB-A-2 206 339.

The content of hydrophobic organic sunscreen(s) present in the particles in accordance with the invention may range from 0.05% to 80% by weight, from 0.1% to 60% by weight and better still from 0.5% to 50% by weight relative to the total weight of the particle.

The content of mineral sunscreens present in the particles may range between 0.05% and 20% by weight and preferentially between 0.1% and 10% by weight relative to the total weight of the particle.

The particles may optionally also contain at least one additional lipophilic active substance, with biological activity that may or may not be related to photoprotection.

The use of calibrated particles in accordance with the invention makes it possible to significantly improve the sun protection factor (SPF) compared with an emulsion whose oily phase is or is not gelled with the same oily gelling agents.

The SPF is advantageously determined according to the “in vitro” method desribed by B. L. Diffey in J. Soc. Cosmet. Chem. 40, 127-133 (1989).

Surfactants

The particles according to the invention may also comprise at least one surfactant.

The presence of (a) surfactant(s) and the chemical nature thereof are generally determined by the nature of the process for preparing the said particles.

Thus, when the particles are prepared according to the process described below, involving an emulsification step, at least one surfactant chosen from nonionic surfactants and ionic surfactants, and mixtures thereof, is introduced into the oily phase-aqueous phase mixture.

The nonionic surfactants, or mixtures thereof, advantageously used in the context of the present invention are surfactants, or mixtures thereof, with an HLB of greater than 5.

As examples of nonionic surfactants that are suitable for use in the invention, mention may be made of:

oxyethylenated or non-oxyethylenated monoalkyl or polyalkyl esters or ethers of glycerol, such as those described in patent U.S. Pat. No. 6,541,018;

oxyethylenated or non-oxyethylenated monoalkyl or polyalkyl esters or ethers of sorbitan, such as those described in U.S. Pat. No. 6,335,022;

monoalkyl or polyalkyl esters or ethers of polyethylene oxide, such as those described in U.S. Pat. No. 6,375,960;

oxyethylenated or non-oxyethylenated monoalkyl or polyalkyl esters or ethers of sugars, such as those described in U.S. Pat. No. 6,689,371; and

mixtures thereof.

The ionic surfactants that may be used in the context of the present invention may be of anionic type, of cationic type or of amphiphilic type.

The anionic surfactants may be chosen especially from:

alkoxylated alkenylsuccinates such as those mentioned in patent U.S. Pat. No. 6,461,625;

alkyl ether citrates such as those mentioned in patent U.S. Pat. No. 6,413,527;

phosphoric alkyl esters such as those described in patent U.S. Pat. No. 6,274,150; and

mixtures thereof.

The alkyl chains of the anionic surfactants that are suitable for use in the invention are advantageously included in the range from C₁₂ to C₂₄, and may be saturated or unsaturated and/or linear or branched.

The ionic surfactants that may be used for the present invention may also be lipoamino acids or alkylsulfonic derivatives, and mixtures thereof.

The lipoamino acids may be chosen especially from monosodium and disodium acylglutamates, for instance the disodium salt of N-stearoyl-L-glutamic acid sold under the name Acylglutamate HS21 by the company Ajinomoto.

The alkylsulfonic derivatives may be chosen especially from the alkylsulfonic derivatives of the first formula below: 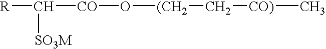
in which R represents an alkyl radical containing from 16 to 22 carbon atoms, and especially a C₁₆H₃₃ or C₁₈H₃₇ radical, taken as a mixture or separately, and M is an alkali metal, for instance sodium.

The cationic surfactants that are suitable for preparing the particles and/or dispersions in accordance with the invention may be chosen especially from quaternary ammonium salts and fatty amines and salts thereof, and mixtures thereof.

The quaternary ammonium salts are, for example:

a) those having the second general formula below: 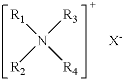
in which the radicals R₁ to R₄, which may be identical or different, represent a linear or branched aliphatic radical containing from 1 to 30 carbon atoms or an aromatic radical such as aryl or alkylaryl.

The aliphatic radicals may comprise heteroatoms especially such as oxygen, nitrogen, sulfur and halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl, containing from about 1 to 30 carbon atoms; X is an anion chosen from the group of halides, phosphates, acetates, lactates, (C₂-C₆)alkyl sulfates, and alkyl- or alkylarylsulfonates.

As quaternary ammonium salts of the second formula presented above, which are advantageously used, mention may be made, firstly, of tetraalkylammonium chlorides, for instance dialkyldimethylammonium or alkyltrimethylammonium chlorides, in which the alkyl radical contains from about 12 to 22 carbon atoms, in particular behenyltrimethylammonium, distearyldimethylaammonium, cetyltrimethylammonium or benzyldimethylstearylammonium chloride, or alternatively, or, secondly, of stearamidopropyldimethyl(myristyl acetate)ammonium chloride sold under the name “Ceraphyl 70” by the company Van Dyk.

b) the quaternary ammonium salts of imidazolinium, for instance those of the third general formula below: 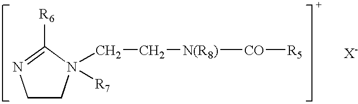
in which R₅ represents an alkenyl or alkyl radical containing from 8 to 30 carbon atoms, for example fatty acid derivatives of tallow; R₆ represents a hydrogen atom, an alkyl radical containing from 1 to 4 carbon atoms or an alkenyl or alkyl radical containing from 8 to 30 carbon atoms; R₇ represents an alkyl radical containing from 1 to 4 carbon atoms; R⁸ represents a hydrogen atom or an alkyl radical containing from 1 to 4 carbon atoms; X is an anion chosen from the group of halides, phosphates, acetates, lactates, alkyl sulfates, and alkyl- or alkylarylsulfonates.

In particular, R₅ and R₆ denote a mixture of alkenyl or alkyl radicals containing from 12 to 21 carbon atoms, for example fatty acid derivatives of tallow, R₇ denotes a methyl radical, R₈ denotes hydrogen. Such a product is sold, for example, under the name “Rewoquat W 75” by the company Rewo.

c) the diquaternary ammonium salts of the fourth general formula below: 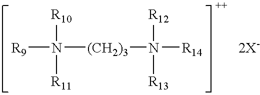
in which R₉ denotes an aliphatic radical containing from about 16 to 30 carbon atoms; R₁₀, R₁₁, R₁₂, R₁₃ and R₁₄ are chosen from hydrogen and an alkyl radical containing from 1 to 4 carbon atoms; and X is an anion chosen from the group of halides, acetates, phosphates, nitrates and methyl sulfates. Such diquaternary ammonium salts especially include propane tallow diammonium dichloride.

As examples of surfactants that are suitable for use in the invention, mention may be made of PEG-30 glyceryl stearate and disodium stearoylglutamate, and mixtures thereof.

According to yet another embodiment variant, the dispersions in accordance with the present invention may comprise as nonionic surfactant a mixture of PEG-30 glyceryl stearate and of disodium stearoylglutamate.

The content of nonionic surfactant and/or ionic surfactant used for the preparation of the particles and/or dispersions in accordance with the invention may advantageously range from 0.5% to 50% by weight, or even from 1% to 40% by weight and in particular from 5% to 20% by weight relative to the total weight of the dispersion.

Emulsifying Polymers

The particles may also contain an emulsifying polymer, i.e. an amphiphilic polymer.

Among the emulsifying polymers that are suitable for use in the invention, mention may be made of:

POE-POP diblock and triblock copolymers such as those described in patent U.S. Pat. No. 6,464,990;

polyoxyethylenated silicone surfactants such as those described in patent U.S. Pat. No. 6,120,778;

non-crosslinked hydrophobic AMPSs such as those described in EP 1 466 588;

amphiphilic acrylic polymers, such as PEMULEN TR-1 or TR-2 or equivalent;

the associative and gelling polymers described in US 2003/0138465;

heat-gelling polymers such as those described in patent applications US 2004/0214913, US 2003/0147832 and US 2002/0198328 and FR 2 856 923.

When they are present, the emulsifying polymer(s) may be introduced in a content ranging from 0.1% to 15% by weight, or even from 0.1% to 10% by weight and more particularly from 0.1% to 5% by weight relative to the total weight of the dispersion.

Process for Obtaining the Dispersions

The structured, calibrated and spherical oily particles in accordance with the invention may be obtained in the form of a dispersion by means of a process comprising at least the steps consisting in:

emulsifying a mixture of at least one sunscreen, at least one oil or an oily phase and at least one oily-phase-gelling polymer with an aqueous and/or water-soluble phase at a temperature above the gel point of the polymer,

subjecting the mixture to a process leading to the production of oily particles, at a temperature at least 5 to 10° C. above the melting point of the mixture used in the preceding step, and

cooling the particle dispersion thus obtained.

It is pointed out that the presence of water in the first step of the process and the execution of the second step with heating are cumulative conditions necessary for obtaining spherical calibrated particles according to the invention.

The viscosity measurement is indeed carried out on the initial mixture, i.e. on the “macrogel” rather than when the particles are already formed, as has already been pointed out hereinabove.

The process according to the invention may, where appropriate, also include a step consisting in diluting the continuous phase of the mixture before the cooling step.

For the purposes of the present invention, the expression “process leading to the production of oily particles” is intended to denote an action of shear type or a mechanism of phase inversion type.

The temperature at which the emulsification step is performed is advantageously greater than 40° C. and advantageously less than 95° C.

Thus, after the process, the dispersions in accordance with the invention comprise in an aqueous and/or water-soluble phase calibrated oily particles comprising at least one sunscreen and comprising an oily phase structured with at least one gelling polymer.

The nature of the process exerted on the oily phase/gelling polymer mixture determines the size of the particles to be obtained.

Thus, for submicron particles, with a mean size of about from 150 nm to 1 μm, it is advantageously possible to use processes that develop a turbulent shear, such as ultrasonication, high-pressure homogenization (working pressure of between 50 and 1000 bar), for example using a Soavi OBL 20® machine from Niro Soavi, or the Microfluidizer® machine from Microfluidics. Processes not requiring any input of mechanical energy may also be used, such as those involving a phase inversion during the emulsification, for instance PIT (phase inversion temperature) or composition inversion (for example by adding a hydrophilic surfactant to a W/O emulsion to invert it to an O/W emulsion).

For the micron-sized particles, with a mean size of about from 1 μm to 20 μm, it is possible to use, for example, processes for obtaining the smallest possible polydispersity, such as the controlled shear of viscoelastic emulsions, as described in patent application FR 2 747 321 and patent U.S. Pat. No. 5,558,820, continuous processes as described in patent U.S. Pat. No. 5,688,842 and patent application WO 02/40574, or those more generally using a colloidal mill, a static mixer, a micromixer, a frame paddle or alternatively a porous membrane, as described in patent U.S. Pat. No. 5,326,484. It is also possible to use processes involving maturation control (U.S. Pat. No. 6,160,061), the swelling of a “templating agent” latex (EP 719 087), Rayleigh instabilities (Weitz, Langmuir, 16, 347-351, (2000)) or fractionation of polydisperse emulsions (Bibette, J. Coll. Int. Sci., vol 147, No. 2, 474-478, (1991)).

In order to facilitate the formation of the particles, during the emulsification step, it is possible, for example, to use one or more nonionic or ionic surfactants and/or hydrophobic emulsifying polymers, as defined above.

Moreover, in the case, for example, of processes developing a laminar shear in order to obtain a uniform particle size distribution, it may optionally be advantageous to adjust the ratio between the viscosity of the dispersed oily phase and the viscosity of the continuous aqueous and/or hydrophilic phase in a ratio ranging from 0.01 to 5 or even from 0.05 to 2. This adjustment may especially be performed by adding surfactants and/or emulsifying polymers such as those described above and/or hydrophilic gelling polymers.

Thus, according to one embodiment of the invention, the aqueous and/or water-soluble continuous phase may also comprise at least one gelling hydrophilic polymer.

Advantageously, when it is present, the gelling hydrophilic polymer is introduced into the aqueous, or hydrophilic, continuous phase in a proportion ranging from 0.01% to 30% by weight and especially from 0.05% to 15% by weight relative to the total weight of the composition.

As examples of gelling hydrophilic polymers, mention may be made especially of carbomers, acrylamidomethylpropanesulfonic (AMPS) derivatives, cellulose derivatives or guar derivatives. As guar derivatives that may advantageously be used in the implementation of the present invention, mention may be made of the hydroxypropyl guar sold under the reference Jaguar HP®105 by the company Rhodia.

Thus, the oily phase-aqueous and/or water-soluble phase mixture may also comprise a compound chosen from a surfactant, an emulsifying polymer, a hydrophilic gelling polymer, and mixtures thereof, and preferably a mixture of a surfactant, an emulsifying polymer and a hydrophilic gelling polymer.

Thus, on account of the process for preparing the particles in accordance with the invention, the particles in accordance with the present invention are advantageously free of volatile solvent.

Dispersion

In accordance with the process for obtaining the particles in accordance with the invention, as described above, these particles are obtained as a dispersion in a continuous and/or water-soluble phase.

The content of calibrated oily particles, comprising at least one sunscreen and comprising an oily phase structured with a gelling polymer, according to the invention, present in the aqueous and/or water-soluble continuous phase may especially be such that the oily mass fraction dispersed in the aqueous and/or water-soluble phase may range from 5% to 89% by weight, especially from 20% to 85% by weight or even from 40% to 80% by weight and in particular from 60% to 80% by weight relative to the total weight of the dispersion.

The structured calibrated oily particles in accordance with the invention advantageously do not aggregate in the dispersion in which they are obtained, and their granulometric specificities in terms of size and distribution index are advantageously conserved therein.

The aqueous and/or water-soluble continuous phase that is suitable for use in the invention may advantageously be water and/or a water-soluble organic solvent, for instance glycols such as glycerol or dipropylene glycol, alone or as mixtures,

For the purposes of the present invention, the term “water-soluble solvent” is intended to denote a compound that is liquid at room temperature and water-miscible (miscibility in water of greater than 50% by weight at 25° C. and at atmospheric pressure).

Among the water-soluble solvents that may be used in the dispersions in accordance with the invention, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones, glycerol and C₂-C₄ aldehydes.

According to yet another embodiment variant, the dispersions in accordance with the present invention may comprise demineralized water as continuous aqueous phase.

According to yet another embodiment variant, the dispersions in accordance with the invention may comprise as nonionic surfactant a mixture of PEG-30 glyceryl stearate and disodium stearoylglutamate, and hydroxypropyl guar as hydrophilic gelling polymer.

According to yet another embodiment variant, the aqueous and/or water-soluble continuous phase that is suitable for use in the invention may contain additional hydrophilic photoprotective agents that are active in the UV-A and/or UV-B range.

Among the hydrophilic organic UV-screening agents that may be used according to the invention, mention may be made of those designated above under their INCI name:

(1) p-aminobenzoic (PABA) derivatives, for instance

PABA,

glyceryl PABA, and

PEG-25 PABA sold under the name “Uvinul P25” by BASF;

(2) benzophenone derivatives comprising at least one sulfonic radical, for instance

benzophenone-4 sold under the trade name “Uvinul MS40” by BASF,

benzophenone-5, and

benzophenone-9;

(3) benzylidenecamphor derivatives comprising at least one sulfonic radical, for instance:

benzylidenecamphorsulfonic acid manufactured under the name “Mexoryl SL” by Chimex,

camphorbenzalkonium methosulfate sold under the name “Mexoryl SO” by Chimex, and

terephthalylidenedicamphorsulfonic acid manufactured under the name “Mexoryl SX” by Chimex;

(4) benzimidazole derivatives comprising at least one sulfonic radical, for instance:

phenylbenzimidazolesulfonic acid sold especially under the trade name “Eusolex 232” by Merck,

bis-benzazolyl derivatives as described in patents EP 669 323 and U.S. Pat. No. 2,463,264 and more particularly the compound disodium phenyldibenzimidazole-tetrasulfonate sold under the trade name “Neo Heliopan AP” by Haarmann & Reimer;

(5) hydrophilic cinnamate derivatives, for instance DEA methoxycinnamate; and

(6) mixtures thereof.

Among these hydrophilic screening agents, the most preferential ones are chosen from

terephthalylidenedicamphorsulfonic acid,

benzophenone-4,

phenylbenzimidazolesulfonic acid,

disodium phenyldibenzimidazoletetrasulfonate,

and also mixtures thereof.

COSMETIC OR DERMATOLOGICAL COMPOSITION

The particles and/or dispersions in accordance with the invention, comprising at least one sunscreen, may advantageously be introduced into various cosmetic and/or dermatological formulations for topical application to the skin and more particularly intended for photoprotection.

Thus, the particles and/or dispersions in accordance with the present invention may be used for the preparation of (a) cosmetic and/or dermatological composition(s) that may be used in the field of photoprotection of keratin materials and more particularly of the skin and the hair.

Thus, a subject of the present invention is also cosmetic or dermatological compositions comprising at least some particles and/or at least one dispersion as defined above. These compositions are useful as products for protecting keratin materials against UV radiation, and in particular the skin.

The compositions comprising particles and/or dispersions in accordance with the invention may be care, hygiene and/or makeup compositions, especially for the skin and the integuments.

In the present case, a composition according to the invention may be in the form of makeup products such as mascaras, eyebrow products, eyeliners, eye shadows, makeup rouges, foundations, lip products, body makeup products, hair makeup products and haircare products such as shampoos, hair conditioners, lotions or gels.

Advantageously, the composition may contain from 0.01% to 40% by weight, especially from 0.1% to 25% by weight or even from 0.2% to 20% by weight of particles in accordance with the present invention relative to the total weight of the composition.

The cosmetic or dermatological composition may be in the form of a lotion, an oil-in-water (O/W) or water-in-oil (W/O) or multiple (W/O/W) emulsion, an aqueous or aqueous-alcoholic gel, a cream, a milk, etc.

Additives

The cosmetic compositions in accordance with the invention may also comprise any additive usually used in the field under consideration, with the proviso that these additives do not impair the property of increasing the sun protection factor of the compositions.

In the context of compositions intended especially for makeup, the additives that may be suitable for use in the invention may be chosen especially from dyestuffs, for instance nacres and pigments, fillers, antioxidants, film-forming agents and, where appropriate, film-forming auxiliaries, essential oils, preserving agents, fragrances, moisturizers, antiseptics and neutralizers, and mixtures thereof.

Needless to say, a person skilled in the art will also take care to select the possible additional additives and/or the amount thereof such that the advantageous properties of the composition according to the invention are not, or are not substantially, adversely affected by the intended addition.

A subject of the present invention is also a non-therapuetic cosmetic makeup and/or care process, comprising at least the step of applying a composition as defined above to the skin.

Finally, a subject of the present invention is the use of particles and/or of at least one dispersion in accordance with the invention for the manufacture of a composition for protecting the skin and/or the hair against the harmful effects of UV radiation, in particular sunlight.

The examples of dispersions of particles and of compositions presented hereinbelow are given as illustrations and with no limiting nature on the invention.

### EXAMPLES

### Examples 1 to 6

### Materials Containing Sunscreens

| | | | | | | 6 |
|---|---|---|---|---|---|---|
| | 1 | 2 | 3 | 4 | 5 | (comparative) |
| Screening agents | | | | | | |
| Butylmethoxydibenzoyl- | 13.1% | 10.1% | 10.8% | 10.1% | 10.1% | 13.1% |
| methane¹ | | | | | | |
| Octocrylene² | 35.6% | 27.4% | 29.2% | 27.4% | 27.4% | 35.6% |
| Oils | | | | | | |
| Dicaprylyl carbonate³ | 26.3% | | 40% | | | 51.3% |
| isopropyl lauroylsarcosinate⁴ | | 37.5% | | | 37.5% | |
| Bis-hydroxyethoxypropyl | | | | 37.5% | | |
| dimethicone⁵ | | | | | | |
| Polymer | | | | | | |
| Ethylenediamine/stearyl | 25% | 25% | | | 12.5% | |
| DimerDilinoleate copolymer⁶ | | | | | | |
| Poly C10-30 alkyl acrylate⁷ | | | 20% | | | |
| Nylon-611/dimethicone | | | | 25% | 12.5% | |
| copolymer (and) PPG-3 | | | | | | |
| myristyl ether⁸ | | | | | | |

¹Parsol 1789 from the company Givaudan
²Uvinul N539 from the company BASF
³Cetiol CC from the company Cognis
⁴Eldew SL-205 from the company Ajinomoto
⁵DC2-5562 Fluid from the company Dow Corning
⁶Uniclear 100VG from the company Arizona Chemical
⁷Doresco IPA 13-6 from the company Landec Corporation
⁸DC2-8178 Gellant from the company Dow Corning

Preparation of the Materials

The screening mixture is homogenized, if necessary, at 80° C. Separately, the polymer is dissolved with stirring, while heating, in the oil, and this mixture is then added to the screening mixture and is homogenized until fully dissolved.

The mixture obtained may be cooled to room temperature and stored or used directly for the manufacture of microparticles.

### Example 7

### Dispersion of the Material of Example 1 in the Form of Microparticles

| | |
|---|---|
| Material obtained according to Example 1 | 40.0% |
| PEG-30 Glyceryl Stearate (Tagat S from the | 1.6% |
| company Degussa) | |
| Disodium Stearoylglutamate | 0.4% |
| (Amisoft HS21P from the company Ajinomoto) | |
| Demineralized water | 58.0% |

The dispersion is prepared in a beaker with an inside diameter of 72 mm comprising a jacket to allow its temperature to be regulated by means of a heating bath. An aqueous solution composed of 6.4 g of PEG-30 glyceryl stearate, 1.6 g of disodium stearoylglutamate and 32 g of demineralized water is introduced therein. The mixture is brought to 85° C. Separately, 160 g of the mixture corresponding to the material 1 of Example 1 are prepared, and this mixture is brought to a temperature of 90° C. It is introduced over 10 minutes into the aqueous surfactant solution while stirring with a paddle 70 mm wide and 2 mm thick at a spin speed of 160 rpm. After 3 minutes, the spin speed is raised to 400 rpm and maintained for 30 minutes.

The emulsion is then diluted with 200 g of demineralized water at 80° C. to achieve a mass fraction of dispersed phase of 40%, and is then cooled to room temperature.

A stable, homogeneous dispersion of microparticles with a granulometry of 1.36 μm (d[v,0.5]) and a uniformity factor (U) of 0.24 is obtained.

### Example 8 (comparative)

### O/W Emulsion Obtained with the Material of Example 6

By applying the procedure of Example 7, with the exception of the spin speed of the paddle, which is 800 rpm, a stable, homogeneous emulsion with a granulometry of 1.63 μm (d[v,0.5]) and a uniformity factor (U) of 0.22 is obtained.

### Example 9

### Microparticle Dispersion Obtained from the Material of Example 2

By applying the procedure of Example 7, a stable, homogeneous dispersion of microparticles with a granulometry of 1.07 μm (d[v,0.5]) and a uniformity factor (U) of 0.24 is obtained.

### Example 10

### Microparticle Dispersion Obtained from the Material of Example 3

By applying the procedure of Example 7, a stable, homogeneous dispersion of microparticles with a granulometry of 2.46 μm (d[v,0.5]) and a uniformity factor (U) of 0.42 is obtained.

### Example 11

### Microparticle Dispersion Obtained from the Material of Example 4

By applying the procedure of Example 7, a stable, homogeneous dispersion of microparticles with a granulometry of 2.00 μm (d[v,0.5]) and a uniformity factor (U) of 0.38 is obtained.

### Example 12

### Dispersion of the Material of Example 1 in the Form of Microparticles

| | |
|---|---|
| Material obtained according to Example 1 | 40.0% |
| PEG-30 Glyceryl Stearate (Tagat S from the | 4.3% |
| company Degussa) | |
| Disodium Stearoylglutamate (Amisoft HS21P from | 1.1% |
| the company Ajinomoto) | |
| Demineralized water | 54.6% |

The dispersion is prepared in a beaker with an inside diameter of 72 mm comprising a jacket to allow its temperature to be regulated by means of a heating bath. An aqueous solution composed of 12.8 g of PEG-30 glyceryl stearate, 3.2 g of disodium stearoylglutamate and 64 g of demineralized water is introduced therein. The mixture is brought to 85° C. Separately, 120 g of the mixture corresponding to the material of Example 1 are prepared, and this mixture is brought to a temperature of 90° C. It is introduced over 10 minutes into the aqueous surfactant solution while stirring with a paddle 70 mm wide and 2 mm thick with a spin speed of 160 rpm. After 3 minutes, the spin speed is raised to 250 rpm and maintained for 30 minutes.

The emulsion is then diluted with 100 g of demineralized water at 80° C. to achieve a mass fraction of dispersed phase of 40%, and is then cooled to room temperature.

A stable, homogeneous dispersion of microparticles with a granulometry of 8.97 μm (d[v,0.5]) and a uniformity factor (U) of 0.35 is obtained.

### Example 13

### Microparticles Containing Sunscreens

The dispersion is prepared in a beaker with an inside diameter of 72 mm comprising a jacket to allow its temperature to be regulated by means of a heating bath. An aqueous solution composed of 1.6 g of PEG-30 glyceryl stearate, 0.1 g of disodium stearoylglutamate, 2.3 g of hydroxyethylcellulose and 116 g of demineralized water is introduced therein. The mixture is brought to 85° C. Separately, 80 g of the following mixture are prepared: 24 g of ethylenediamine/stearyl dimerdilinoleate copolymer (Uniclear 100VG from the company Arizona Chemical), 19.6 g of dicaprylyl carbonate (Cetiol CC from the company Cognis), 26.6 g of octocrylene (Uvinul N539 from the company BASF) and 9.8 g of butylmethoxydibenzoylmethane (Parsol 1789 from the company Givaudan), and this mixture is brought to a temperature of 90° C.

This material is introduced over 10 minutes into the aqueous surfactant solution while stirring with a paddle 70 mm wide and 2 mm thick with a spin speed of 300 rpm. After 3 minutes, the spin speed is raised to 600 rpm and maintained for 30 minutes.

The emulsion is then cooled to room temperature.

A stable, homogeneous dispersion of microparticles having the composition below is obtained:

| | |
|---|---|
| Gelled sunscreens | 40.00% |
| PEG-30 Glyceryl Stearate (Tagat S from the | 0.80% |
| company Degussa) | |
| Disodium Stearoylglutamate (Amisoft HS21P from | 0.05% |
| the company Ajinomoto) | |
| Hydroxyethylcellulose (Natrosol 250HHR from the | 1.15% |
| company Aqualon) | |
| Demineralized water | 58.00% |

The dispersion has a granulometry of 11.3 μm (d[v,0.5]) and a uniformity factor (U) of 0.30.

### Example 14

### Microparticles Containing Sunscreens

The dispersion is prepared in a beaker with an inside diameter of 72 mm comprising a jacket to allow its temperature to be regulated by means of a heating bath. An aqueous solution composed of 2.28 g of PEG-30 glyceryl stearate, 0.12 g of disodium stearoylglutamate, 1.8 g of hydroxypropyl guar (Jaguar HP105 from the company Rhodia) and 115.8 g of demineralized water is introduced therein. The mixture is brought to 85° C. Separately, 80 g of the material of Example 1 are prepared, and this material is brought to a temperature of 90° C.

This material is introduced over 10 minutes into the aqueous surfactant solution while stirring with a paddle 70 mm wide and 2 mm thick with a spin speed of 300 rpm. After 3 minutes, the spin speed is raised to 600 rpm and maintained for 30 minutes.

The emulsion is then cooled to room temperature.

A stable, homogeneous dispersion of microparticles having the composition below is obtained:

| | |
|---|---|
| Gelled sunscreens | 40.00% |
| PEG-30 Glyceryl Stearate (Tagat S from the | 1.14% |
| company Degussa) | |
| Disodium Stearoylglutamate (Amisoft HS21P from the | 0.06% |
| company Ajinomoto) | |
| Hydroxypropyl guar (Jaguar HP105 from the company | 0.90% |
| Rhodia) | |
| Demineralized water | 57.90% |

The dispersion has a granulometry of 7.4 μm (d[v,0.5]) and a uniformity factor (U) of 0.40.

### Example 15

### Microparticles Containing Sunscreens

The dispersion is prepared in a beaker with an inside diameter of 72 mm comprising a double jacket to allow its temperature to be regulated by means of a heating bath. An aqueous solution composed of 12 g of polyvinyl alcohol (Celvol 203 from the company Celanese) and 78 g of demineralized water is introduced therein. The mixture is brought to 85° C. Separately, 120 g of the material of Example 1 are prepared, and this material is brought to a temperature of 90° C.

This material is introduced over 10 minutes into the aqueous surfactant solution while stirring with a paddle 70 mm wide and 2 mm thick, with a spin speed of 160 rpm. After 3 minutes, the spin speed is raised to 400 rpm and maintained for 30 minutes.

The emulsion is then cooled to room temperature.

A stable, homogeneous dispersion of microparticles having the composition below is obtained:

| | |
|---|---|
| Gelled sunscreens | 40.00% |
| Polyvinyl alcohol (Celvol 203 from | 4.00% |
| the company Celanese) | |
| Demineralized water | 56.00% |

The dispersion has a granulometry of 6.85 μm (d[v,0.5]) and a uniformity factor (U) of 0.40.

### Example 16

### Nanoparticles Containing Sunscreens

| | |
|---|---|
| Material obtained according to Example 2 | 20.00% |
| PEG-30 Glyceryl Stearate (Tagat S from the company | 1.80% |
| Degussa) | |
| Disodium Stearoylglutamate (Amisoft HS21P from the | 0.20% |
| company Ajinomoto) | |
| Demineralized water | 78.00% |

An aqueous solution composed of 9 g of PEG-30 glyceryl stearate, 1 g of disodium stearoylglutamate and 390 g of demineralized water is prepared. The mixture is brought to 85° C. Separately, 100 g of the mixture corresponding to material 2 are prepared, and this mixture is brought to a temperature of 90° C. It is introduced into the aqueous surfactant solution while stirring with a device of rotor-stator type (Ultra-Turrax T50). After 5 minutes, the mixture is homogenized at 85° C. using a Soavi Panda homogenizer, by homogenization twice at a pressure of 400 bar.

The dispersion is then cooled to room temperature.

A stable, homogeneous dispersion of nanoparticles with a mean diameter of 300 nm is obtained.

The SPF (in vitro) of the compositions below are compared:

### Example 17

| | |
|---|---|
| Emulsion of Example 8 | 50.00% |
| Hydroxypropyl guar (Jaguar HP105 from the | 0.50% |
| company Rhodia) | |
| Demineralized water | 49.50% |

### Example 18

| | |
|---|---|
| Dispersion of Example 7 | 50.00% |
| Hydroxypropyl guar (Jaguar HP105 from the | 0.50% |
| company Rhodia) | |
| Demineralized water | 49.50% |

The sun protection factor (SPF) is determined according to the “in vitro” method described by B. L. Diffey in J. Soc. Cosmet. Chem. 40, 127-133, (1989).

The measurements were taken using a UV-1000S spectrophotometer from the company Labsphere. Each composition is applied to a Transpore adhesive strip from 3M bonded to a quartz slide, in the form of a homogeneous and uniform deposit at a rate of 1 mg/cm².

SPF Measurements

| | SPF | error |
|---|---|---|
| Example 17 | 6.9 | 0.13 |
| Example 18 | 32.2 | 0.29 |

It is found that the particles of the invention make it possible to improve the sun protection factor when compared with an emulsion of similar granulometry not containing any crystalline polymer.

The SPF of the compositions below is also compared:

### Example 19 (comparative)

### O/W Emulsion without Crystalline Polymer

| Phase A | |
|---|---|
| PEG-20 methylglucose sesquistearate (GLUCAMATE | 2.00% |
| SSE 20 from Chemron) | |
| Disodium EDTA | 0.10% |
| Glycerol | 5.00% |
| Preserving agent | 0.20% |
| Demineralized water | 56.70% |
| Phase B | |
| Methylglucose sesquistearate | 2.00% |
| Stearyl alcohol (and) Ceteareth-20 | 2.00% |
| Cyclohexasiloxane | 5.85% |
| C12-15 alkyl benzoate | 5.85% |
| Dicaprylyl carbonate | 2.62% |
| Butylmethoxydibenzoylmethane | 1.32% |
| Octocrylene | 3.56% |
| Ethylenediamine/stearyl dimerdilinoleate copolymer | 2.50% |
| (Uniclear 100VG from the company Arizona Chemical) | |
| Preserving agent | 0.10% |
| Phase C | |
| Polyacrylamide (and) C13-14 isoparaffin (and) | 1.00% |
| Laureth-7 (Sepigel 305 from SEPPIC) | |
| Ammonium polyacryloyldimethyl taurate | 1.20% |
| (Hostacerin AMPS from Clariant) | |
| Phase D | |
| Biosaccharide Gum-1 (Fucogel 1000PP from Solabia) | 5.00% |
| Phase E | |
| Aluminium starch octenylsuccinate | 3.00% |

### Example 20 (comparative)

### O/W Emulsion with Crystalline Polymer

Phase A

| | |
|---|---|
| PEG-20 methylglucose sesquistearate (GLUCAMATE | 2.00% |
| SSE 20 from Chemron) | |
| Disodium EDTA | 0.10% |
| Glycerol | 5.00% |
| Preserving agent | 0.20% |
| Demineralized water | 56.70% |

Phase B

| | |
|---|---|
| Methylglucose sesquistearate | 2.00% |
| Stearyl alcohol (and) Ceteareth-20 | 2.00% |
| Cyclohexasiloxane | 7.10% |
| C12-15 alkyl benzoate | 7.10% |
| Dicaprylyl carbonate | 2.62% |
| Butylmethoxydibenzoylmethane | 1.32% |
| Octocrylene | 3.56% |
| Preserving agent | 0.10% |

Phase C

| | |
|---|---|
| Polyacrylamide (and) C13-14 isoparaffin (and) | 1.00% |
| Laureth-7 (Sepigel 305 from SEPPIC) | |
| Ammonium polyacryloyldimethyl taurate (Hostacerin | 1.20% |
| AMPS from Clariant) | |

Phase D

| | |
|---|---|
| Biosaccharide Gum-1 (Fucogel 1000PP from | 5.00% |
| Solabia) | |

Phase E

| | |
|---|---|
| Aluminium starch octenylsuccinate | 3.00% |

### Example 21

### O/W Emulsion Containing the Microparticles of the Invention

Phase A

| | |
|---|---|
| PEG-20 methylglucose sesquistearate (GLUCAMATE | 2.00% |
| SSE 20 from Chemron) | |
| Disodium EDTA | 0.10% |
| Glycerol | 5.00% |
| Preserving agent | 0.20% |
| Demineralized water | 41.70% |

Phase B

| | |
|---|---|
| Methylglucose sesquistearate | 2.00% |
| Stearyl alcohol (and) Ceteareth-20 | 2.00% |
| Cyclohexasiloxane | 5.85% |
| C₁₂₋₁₅ alkyl benzoate | 5.85% |
| Preserving agent | 0.10% |

Phase C

| | |
|---|---|
| Polyacrylamide (and) C13-14 isoparaffin (and) | 1.00% |
| Laureth-7 (Sepigel 305 from SEPPIC) | |
| Ammonium polyacryloyldimethyl taurate (Hostacerin | 1.20% |
| AMPS from Clariant) | |

Phase D

| | |
|---|---|
| Biosaccharide Gum-1 (Fucogel 1000PP from Solabia) | 5.00% |

Phase E

| | |
|---|---|
| Aluminum starch octenylsuccinate | 3.00% |

Phase F

| | |
|---|---|
| Microparticle dispersion of Example 7 | 25.00% |

SPF Measurements

| | SPF | Error |
|---|---|---|
| Example 19 | 10.4 | 0.24 |
| (comparative) | | |
| Example 20 | 12.2 | 0.19 |
| (comparative) | | |
| Example 21 | 22.8 | 0.18 |

In the light of these results, an increase in the SPF is observed when an oil-gelling agent is introduced into the fatty phase of an emulsion. Furthermore, this increase in the SPF is potentiated when the sunscreens are introduced into the emulsion in the form of calibrated microparticles according to the invention.

What is claimed is:

1. Calibrated and spherical oily particles, comprising:
at least one hydrophobic sunscreen;
at least one oily phase structured with at least one gelling polymer;
wherein:
the particles have a mean size of less than or equal to 20 μm;
the structured oily phase has a melting point of greater than or equal to 40° C.; and
the particles have a circularity index of from 0.9 to 1.

2. The particles of claim 1, wherein the mean size is less than or equal to 12 μm.

3. The particles of claim 1, wherein the mean size is from 150 nm to 12 μm.

4. The particles of claim 1, wherein the structured oily phase has a melting point of from 50° C. to 90° C.

5. The particles of claim 1, wherein when the particles have a mean micrometric size, the particles have a coefficient of uniformity of less than or equal to 0.45.

6. The particles of claim 1, wherein when the particles have a mean submicrometric size, the particles have a polydispersity index of less than or equal to 0.35.

7. The particles of claim 1, wherein the particles are substantially spherical.

8. The particles of claim 1, wherein the oily phase comprises at least one non-volatile oil.

9. The particles of claim 1, wherein the oily phase is present in an amount of from 20% to 99% by weight relative to a total weight of the particles.

10. The particles of claim 1, wherein the oily phase comprises at least one oil selected from the group consisting of a plant oil, an animal oil, a synthetic oil and a mineral oil.

11. The particles of claim 1; wherein the gelling polymer comprises at least one polymer selected from the group consisting of a semi-crystalline polymer, a polyamide, a silicone polyamide, a polysaccharide monoalkyl ester, a polysaccharide polyalkyl ester, and a diblock and/or triblock and/or multiblock and/or radial-block copolymer.

12. The particles of claim 1, wherein the gelling polymer comprises at least one member selected from the group consisting of ethylenediamine/stearyl dimerdilinoleate and a semi-crystalline polymer of poly-C₁₀₋₃₀ alkyl acrylate type.

13. The particles of claim 1, wherein the gelling polymer is present in an amount of from 1% to 60% by weight relative to a total weight of the particles.

14. The particles of claim 1, wherein a weight ratio of the gelling polymer to the oily phase of the particles is from 0.1 to 1.

15. The particles of claim 1, wherein the hydrophobic sunscreen comprises at least one member selected from the group consisting of hydrophobic organic photoprotective agents and mineral photoprotective agents.

16. The particles of claim 1, wherein the hydrophobic sunscreen comprise at least one member selected from the group consisting of anthranilates, cinnamic derivatives, dibenzoylmethane derivatives, salicylic derivatives, camphor derivatives, triazine derivatives, benzophenone derivatives, β,β-diphenyl acrylate derivatives, benzotriazole derivatives, benzalmalonate derivatives, benzimidazole derivatives, imidazolines, bis-benzazolyl derivatives, p-aminobenzoic acid (PABA) derivatives, methylenebis(hydroxyphenylbenzotriazole) derivatives, screening polymers and screening silicones, dimers derived from α-alkylstyrene, and 4,4-diarylbutadienes.

17. The particles of claim 1, wherein the hydrophobic sunscreen is present in an amount of from 0.5% to 50% by weight relative to a total weight of the particles.

18. An aqueous and/or water-soluble phase dispersion, comprising the particles of claim 1.

19. The dispersion of claim 18, further comprising at least one surfactant selected from the group consisting of an ionic surfactant and a nonionic surfactant.

20. The dispersion of claim 18, further comprising at least one hydrophobic emulsifying polymer.

21. The dispersion of claim 18, further comprising at least one hydrophilic gelling polymer.

22. The dispersion of claim 18, wherein the particles are dispersed in an aqueous phase, and the particles represent an oily mass fraction of from 60% to 80% by weight relative to a total weight of the dispersion.

23. A cosmetic or dermatological composition, comprising the particles of claim 1.

24. A method of forming a photoprotective cosmetic composition, comprising employing the particles of claim 1.

25. A non-therapeutic process for making up and/or caring for the skin, comprising applying a composition comprising the particles of claim 1 to the skin.

26. A method of manufacturing a composition for protecting the skin and/or the hair against the harmful effects of UV radiation comprising employing the particles of claim 1.

27. A process for manufacturing a dispersion of calibrated and spherical oily particles comprising at least one sunscreen and at least one oily phase structured with at least one gelling polymer and having a mean size of less than or equal to 20 μm, the process comprising:
emulsifying a mixture of at least one sunscreen, at least one oil or an oily phase and at least one oily-phase-gelling polymer with an aqueous and/or water-soluble phase at a temperature above a gel point of the polymer;
subjecting the mixture to a process leading to the production of the oily particles at a temperature at least 5° C. above a melting point of the mixture; and
cooling the obtained particle dispersion.

28. The process of claim 27, wherein the gelling polymer comprises at least one polymer selected from the group consisting of a semi-crystalline polymer, a polyamide, a silicone polyamide, a polysaccharide monoalkyl ester, a polysaccharide polyalkyl ester, and a diblock and/or triblock and/or multiblock and/or radial-block copolymer.

29. The process of claim 27, wherein the gelling polymer comprises at least one member selected from the group consisting of ethylenediamine/stearyl dimerdilinoleate and a semi-crystalline polymer of poly-C₁₀₋₃₀ alkyl acrylate type.

30. The process of claim 27, wherein the gelling polymer is present in an amount of from 1% to 80% by weight relative to a total weight of the particles.

31. The process of claim 27, wherein particles obtained by the process have a mean size of from about 1 to about 15 micrometers.

32. The process of claim 27, wherein the process employs at least one technique selected from the group consisting of controlled shear of viscoelastic emulsions, a process using a colloidal mill, a process using a static mixer, a process using a micromixer, a process using a frame paddle, a process using an extruder-blender, a process using a porous membrane, a process using maturation control, a process using swelling of a “templating agent” latex, a process using Rayleigh instabilities, and a process using fractionation of a polydisperse emulsion.

33. The process of claim 27, wherein particles obtained by the process have a mean size of from about 150 nm to about 1 μm.

34. The process of claim 27, wherein the process employs at least one technique selected from the group consisting of a process developing a shear and a process involving a phase inversion.

35. The process of claims 27, wherein the temperature above the gel point of the polymer is greater than 40° C.

36. A dispersion of calibrated and spherical oily particles prepared by the process of claim 27.

37. Calibrated and spherical oily particles, comprising:
at least one sunscreen; and
at least one oily phase structured with at least one gelling polymer;
wherein:
the particles have a mean size of less than or equal to 20 μm;
the gelling polymer is at least one member selected from the group consisting of a semi-crystalline polymer, a polyamide, a silicone polyamide, a polysaccharide monoalkyl ester or polyalkyl ester, and a diblock and/or triblock and/or multiblock and/or radial-block copolymer; and
the particles have a circularity index of from 0.9 to 1.

38. The particles of claim 37, wherein the mean size is from 150 nm to 12 μm.

39. The particles of claim 37, wherein the structured oily phase has a melting point of from 50° C. to 90° C.

40. The particles of claim 37, wherein when the particles have a mean micrometric size, the particles have a coefficient of uniformity of less than or equal to 0.45.

41. The particles of claim 37, wherein when the particles have a mean submicrometric size, the particles have a polydispersity index of less than or equal to 0.35.

42. The particles of claim 37, wherein the oily phase comprises at least one non-volatile oil.

43. The particles of claim 37, wherein the oily phase is present in an amount of from 20% to 99% by weight relative to a total weight of the particles.

44. The particles of claim 37, wherein the oily phase comprises at least one oil selected from the group consisting of a plant oil, an animal oil, a synthetic oil and a mineral oil.

45. The particles of claim 37, wherein the gelling polymer comprises at least one member selected from the group consisting of ethylenediamine/stearyl dimerdilinoleate and a semi-crystalline polymer of poly-C₁₀₋₃₀ alkyl acrylate type.

46. The particles of claim 37, wherein the gelling polymer is present in an amount of from 1% to 60% by weight relative to a total weight of the particles.

47. The particles of claim 37, wherein a weight ratio of the gelling polymer to the oily phase of the particles is from 0.1 to 1.

48. The particles of claim 37, wherein the hydrophobic sunscreen is present in an amount of from 0.5% to 50% by weight relative to a total weight of the particles.

49. An aqueous and/or water-soluble phase dispersion, comprising the particles of claim 37.

50. A cosmetic or dermatological composition, comprising the particles of claim 37.

51. A method of forming a photoprotective cosmetic composition, comprising employing the particles of claim 37.

52. A non-therapeutic process for making up and/or caring for the skin, comprising applying a composition comprising the particles of claim 37 to the skin.

53. A method of manufacturing a composition for protecting the skin and/or the hair against the harmful effects of UV radiation comprising employing the particles of claim 37.

54. Calibrated and spherical oily particles, comprising at least one sunscreen, wherein:
the particles are obtained from an oily phase structured with at least one gelling polymer;
the gelling polymer is of a nature and/or is present in an amount sufficient to give the oily phase a viscosity of greater than or equal to 750 Pa·s at a shear of 1 s⁻¹ at 25° C.; and
the particles have a circularity index of from 0.9 to 1.

55. The particles of claim 54, wherein the mean size is from 150 nm to 12 μm.

56. The particles of claim 54, wherein the structured oily phase has a melting point of from 50° C. to 90° C.

57. The particles of claim 54, wherein when the particles have a mean micrometric size, the particles have a coefficient of uniformity of less than or equal to 0.45.

58. The particles of claim 54, wherein when the particles have a mean submicrometric size, the particles have a polydispersity index of less than or equal to 0.35.

59. The particles of claim 54, wherein the oily phase comprises at least one non-volatile oil.

60. The particles of claim 54, wherein the oily phase is present in an amount of from 20% to 99% by weight relative to a total weight of the particles.

61. The particles of claim 54, wherein the oily phase comprises at least one oil selected from the group consisting of a plant oil, an animal oil, a synthetic oil and a mineral oil.

62. The particles of claim 54, wherein the gelling polymer comprises at least one polymer selected from the group consisting of a semi-crystalline polymer, a polyamide, a silicone polyamide, a polysaccharide monoalkyl ester, a polysaccharide polyalkyl ester, and a diblock and/or triblock and/or multiblock and/or radial-block copolymer.

63. The particles of claim 54, wherein the gelling polymer comprises at least one member selected from the group consisting of ethylenediamine/stearyl dimerdilinoleate and a semi-crystalline polymer of poly-C¹⁰⁻³⁰ alkyl acrylate type.

64. The particles of claim 54, wherein the gelling polymer is present in an amount of from 1% to 60% by weight relative to a total weight of the particles.

65. The particles of claim 54, wherein a weight ratio of the gelling polymer to the oily phase of the particles is from 0.1 to 1.

66. The particles of claim 54, wherein the hydrophobic sunscreen is present in an amount of from 0.5% to 50% by weight relative to a total weight of the particles.

67. An aqueous and/or water-soluble phase dispersion, comprising the particles of claim 54.

68. A cosmetic or dermatological composition, comprising the particles of claim 54.

69. A method of forming a photoprotective cosmetic composition, comprising employing the particles of claim 54.

70. A non-therapeutic process for making up and/or caring for the skin, comprising applying a composition comprising the particles of claim 54 to the skin.

71. A method of manufacturing a composition for protecting the skin and/or the hair against the harmful effects of UV radiation comprising employing the particles of claim 54.
